(19) United States
(12) Patent Application Publication
(10) Pub. No.: US 2013/0274303 A1
(43) Pub. Date: Oct. 17, 2013

(54) METHODS AND COMPOSITIONS FOR TREATMENT OF DRUG ADDICTION

(71) Applicants: EMORY UNIVERSITY, (US); YALE UNIVERSITY, (US)

(72) Inventors: David WEINSHENKER, Atlanta, GA (US); Robert T. MALISON, Guilford, CT (US)

(73) Assignees: YALE UNIVERSITY, New Haven, CT (US); EMORY UNIVERSITY, Atlanta, GA (US)

(21) Appl. No.: 13/633,397

(22) Filed: Oct. 2, 2012

Related U.S. Application Data
(63) Continuation of application No. 12/530,471, filed on Sep. 9, 2009, now abandoned.
(60) Provisional application No. 60/895,224, filed on Mar. 16, 2007.

(51) Int. Cl. A61K 31/417

(52) U.S. Cl. CPC A61K 31/417
USPC 514/392

(57) ABSTRACT

The present disclosure relates to methods of treating a stimulant addiction of a patient comprising administering to a patient in need a therapeutically effective dose of a selective dopamine beta-hydroxylase inhibitor thereby decreasing stimulant reward, inducing aversion for the stimulant or preventing relapse in the patient. The disclosure further encompasses methods whereby a therapeutically effective dose of a selective dopamine beta-hydroxylase inhibitor is determined by: characterizing the genetic profile of the patient with respect to the gene encoding dopamine beta-hydroxylase, a polymorphism therein correlating to the level of endogenous dopamine beta-hydroxylase activity in the patient before administering the therapeutic agent.

### CROSS REFERENCE TO RELATED APPLICATIONS

This application is a continuation and claims priority to U.S. patent application Ser. No. 12/530,471 filed Sep. 9, 2009, and claims priority to PCT Application Number PCT/US08/56024, filed September Mar. 6, 2008, and U.S. Provisional Application entitled “Methods and Compositions for Treatment of Drug Addiction” Ser. No. 60/895,224 filed Mar. 16, 2007, all the contents of the related applications are hereby expressly incorporated herein by reference in their entirety.

### STATEMENT REGARDING FEDERALLY SPONSORED RESEARCH

This invention was made with government support under NIH Grants Nos. 5T32DA015040-02 and 1RO3DA019849-01 awarded by the U.S. National Institutes of Health of the United States government. The government has certain rights in the invention

### FIELD

The present disclosure relates to compositions and methods of use thereof for treating stimulant addiction in a patient. The disclosure further relates to methods of determining effective doses of compounds for the treatment of stimulant addiction.

### BACKGROUND

Drug addiction represents a serious problem for many individuals, their families and society in general. While treatment for substance abuse and dependence often focuses on combating the psychological aspects of addiction, patients in treatment also often receive prescription drugs to assist in their recovery in a variety of ways. Finding new treatments to help addicts overcome their addiction and avoid future drug use would provide a significant advantage in combating drug addiction.

Cocaine is a widely abused psychostimulant drug that acts by blocking the plasma membrane transporters for dopamine, norepinephrine (NE), and serotonin. In humans, cocaine use results in a broad spectrum of effects, both subjectively positive (e.g., euphoria, increased energy, enhanced alertness) and negative (e.g., anxiety, paranoia, nausea, hypertension). In addition to its well-documented rewarding and locomotor activating effects in rodents, cocaine also induces anxiety-like behavior that can be reversed by administration of typical anxiolytic drugs, such as diazepam (Ettenberg & Geist (1991) Psychopharmacol. 103: 455-461; Rogiero & Takahashi (1992) Pharmacol., Biochem. Behavior 43: 631-633; Yang et al., (1992) Pharmacol. Biochem. Behavior 41: 643-650; Costall et al., (1988) Pharmacol. Biochem. Behavior 33: 197-203; Blanchard & Blanchard (1999) Neurosci. & Biobehavioral Revs. 23: 981-991; David et al., (2001) Neuropsychopharmacol. 24(3): 300-318; Paine et al., (2002) Behavioural Pharmacol. 13: 511-523).

Whereas cocaine-induced reward has been studied extensively, less is known about the processes underlying the negative behavioral states associated with acute administration of the drug. Although dopamine signaling has been primarily implicated in psychostimulant responses, cocaine also increases extracellular NE levels, and NE transmission has been shown to modulate psychostimulant-induced behaviors and neurochemistry (Drouin et al., (2002) J. Neurosci. 22: 2873-2884; Schank et al., (2006) Neuropsychopharmacol, 31: 2221-2230; Ventura et al., (2003) J. Neurosci. 23:1879-1885; Weinshenker et al., (2002) Proc. Natl. Acad. Sci. USA 99(21): 13873-13877). Given that NE modulates general stress and anxiety responses (Gorman & Dunn (1993) Pharmacol. Biochem. & Behavior 45: 1-7; Stanford S. C (1995) Pharmacol. & Therapeut. 68: 297-342), it was surmised that NE might also play a critical role in cocaine-induced anxiogenesis.

Dopamine beta-hydroxylase (DBH) is the enzyme that converts dopamine to norepinephrine in the catecholamine biosynthetic pathway, and therefore Dbh knockout (Dbh −/−) mice lack NE completely (Thomas et al (1995) Nature 374:643-646; Thomas et al., (1998) J. Neurochem. 70: 2468-2476). It has been shown that Dbh −/− mice exhibit an increase in striatal high affinity-state DA receptors and a corresponding hypersensitivity to the locomotor activating, rewarding, and aversive effects of cocaine (Schank et al., (2006) Neuropsychopharmacol, 31: 2221-2230). In particular, a novel cocaine-induced place aversion was observed in Dbh −/− mice at a dose of 20 mg/kg, a dose that produces a robust place preference in control animals.

The compound disulfuram (tetraethylthiuram; ANTABUSE™) has been used for over 50 years in the treatment of alcoholism (Fuller et al., (1986) JAMA 256: 1449-1455). Disulfuram inhibits the enzyme aldehyde dehydrogenase, which results in accumulation of the toxic metabolic intermediate acetaldehyde upon ethanol ingestion. Acetaldehyde produces the “Antabuse reaction”, an aversive syndrome consisting of flushing, nausea, and vomiting. Avoidance of this syndrome by reducing alcohol intake is believed to be responsible for the reductions in alcohol use in dependent individuals. More recently, disulfuram has also been used to treat cocaine dependence; however, the exact mechanism of action was unknown, since accumulation of acetaldehyde does not occur in cocaine users who do not use alcohol. Additionally, disulfuram results in some undesirable side-effects. The development of new compounds and pharmaceutical compositions, therefore, specifically directed at the treatment of stimulant addiction would be advantageous.

### SUMMARY

In general, the present disclosure relates to methods of treating stimulant addiction, and most advantageously of treating a cocaine addiction, by specifically inhibiting the dopamine beta-hydroxylase enzyme. One aspect of the present disclosure encompasses methods of treating a stimulant addiction of a patient, comprising: administering to a patient in need of treatment for stimulant addiction a therapeutically effective dose of a composition comprising a selective dopamine beta-hydroxylase inhibitor. Although it is not intended that the claimed embodiments be limited by any particular mechanism, the therapeutic dose may act via one or more of three mechanisms: (1) decreases the rewarding effects of the stimulant in the patient, (2) increases the aversive effects of the stimulant in the patient, or (3) attenuates relapse caused by drug re-exposure, stress, or drug-associated cues after a period of abstinence. In certain embodiments, the methods of treatment of the present disclosure advantageously use therapeutic agents specifically targeting dopamine beta-hydroxylase, thereby reducing or eliminating side-effects that arise from using less specific agents such as disulfuram.

In embodiments of this aspect of the disclosure, the selective dopamine beta-hydroxylase inhibitor may be, but is not limited to, a compound having a formula selected from Formulas I, II, III, IV, (S)-5,7,-difluoro-1,2,3,4-tetrahydronaphthalen-2-ylamine, and nepicastat (S-5-amino methyl-1-(5,7-difluoro-1,2,3,4-tetrahydronaphthalen-2-yl)-1,3-dihydroimidazole-2-thione hydrochloride), or a derivative of each, or a pharmaceutically acceptable salt of each.

In one embodiment of the methods of the disclosure, the composition may comprise the selective dopamine beta-hydroxylase inhibitor nepicastat (S-5-aminomethyl-1-(5,7-difluoro-1,2,3,4-tetrahydronaphthalen-2-yl)-1,3-dihydroimidazole-2-thione hydrochloride).

In embodiments of the methods of the disclosure, it is contemplated that the composition administered to the patient in need thereof may further comprise a pharmaceutically acceptable carrier and, optionally, other therapeutic agents that may be useful to alleviate adverse symptoms of the stimulant addiction or side-effects of the administered treatment.

This aspect of the disclosure, therefore, provides methods of treating a stimulant addiction of a patient, wherein the patient is addicted to cocaine or a derivative thereof, or to an amphetamine or a derivative thereof. The methods of the disclosure are especially advantageous for treating addictions due to agents such as cocaine that increase extracellular norepinephrine.

In one embodiment of the methods, the stimulant addiction is cocaine addiction.

The present disclosure further encompasses methods of generating abstinence from an addictive compound by administering to a patient having an addiction to a stimulant, an amount of a therapeutic composition comprising a selective dopamine beta-hydroxylase inhibitor, wherein the amount administered is effective in generating a response in the recipient patient such that the recipient develops (1) a decrease in the rewarding properties of the stimulant, (2) an aversion to the intake of the stimulant, or (3) an attenuation of relapse precipitated by pharmacological or environmental factors.

These methods of the disclosure are especially useful in the treatment of a patient addicted to cocaine or a derivative thereof, or to an amphetamine or a derivative thereof.

In one embodiment of the disclosure, the stimulant addiction is cocaine addiction or addiction to a derivative thereof.

In embodiments of this method of the disclosure, the selective dopamine beta-hydroxylase inhibitor can be nepicastat.

In certain embodiments, this disclosure also provides methods of treating a stimulant addiction of a patient, wherein the therapeutically effective dose administered to the patient is selected by: determining the genetic profile of a patient with respect to the gene encoding dopamine beta-hydroxylase, wherein the genetic profile correlates to the level of dopamine beta-hydroxylase activity in the patient; and determining a therapeutically effective dosage of a selective dopamine beta-hydroxylase inhibitor according to the genetic profile of the dopamine beta-hydroxylase encoding gene.

In embodiments of these methods of the disclosure when the patient is homozygous negative for dopamine beta-hydroxylase, the therapeutically effective dose administered to the patient may be advantageously less than if the patient has at least one dopamine beta-hydroxylase positive allele.

Another aspect of the disclosure encompasses methods of selecting a therapeutic dose of a composition for treatment of a patient having a stimulant addiction comprising: determining the genetic profile of a patient with respect to a gene encoding dopamine beta-hydroxylase, wherein the genetic profile correlates to the level of dopamine beta-hydroxylase activity in the patient; and determining a therapeutically effective dosage of a selective dopamine beta-hydroxylase inhibitor according to the genetic profile of the dopamine beta-hydroxylase encoding gene.

### BRIEF DESCRIPTION OF THE FIGURES

The disclosure can be better understood with reference to the following drawings.

FIG. 1 illustrates embodiments of a compound of Formula I, and derivatives thereof.

FIG. 2 illustrates embodiments of a compound of Formula II, and derivatives thereof.

FIG. 3 illustrates embodiments of a compound of Formula III, and derivatives thereof.

FIG. 4 illustrates the structure of (S)-5,7,-difluoro-1,2,3,4-tetrahydronaphthalen-2-ylamine.

FIG. 5 illustrates the structure of the selective DBH inhibitor Nepicastat (S-5-amino methyl-1-(5,7-difluoro-1,2,3,4-tetrahydronaphthalen-2-yl)-1,3-dihydroimidazole-2-thione hydrochloride).

FIG. 6 illustrates the effects of cocaine-induced locomotion in Dbh −/− mice.

FIG. 7 illustrates the altered cocaine reward and aversion in Dbh −/− mice.

FIG. 8 illustrates the effect of the highly specific DBH inhibitor, nepicastat, on drug-induced behavior in mice.

FIG. 9 illustrates that a single, acute dose of nepicastat does not significantly affect cocaine-induced locomotion.

FIG. 10 illustrates that the effect of chronic DBH inhibition by nepicastat recapitulates the cocaine hypersensitivity previously observed in DBH knockout mice.

FIG. 11 illustrates the effects of cocaine on performance in the elevated plus maze in Dbh +/− and Dbh −/− knockout mice. Cocaine was administered to mice 20 minutes prior to the EPM. Shown is percent open arm time during the five minute test. **p<0.01 compared to vehicle control for that genotype. **p<0.01, ***p<0.001 compared to Dbh +/− mice for that dose (N=8 per group).

FIG. 12 illustrates that disulfuram attenuates cocaine-induced anxiety in Dbh +/− mice. Dbh +/− mice were injected with disulfuram (3 times 200 mg/kg, i.p., two hours between each injection) or vehicle. Two hours following the last disulfuram treatment, mice were injected with saline or cocaine (10 mg/kg, i.p.), and tested in the EPM 20 minutes later. Shown is percent open arm time during the five minute EPM test. *p<0.05 compared to Vehicle-Saline group (N=8 per group).

FIG. 13 illustrates that the .beta.-adrenergic antagonist propranolol attenuates cocaine-induced anxiety. Dbh +/− mice were treated with vehicle, the alpha1-adrenergic antagonist prazosin, the alpha 2-adrenergic antagonist yohimbine, or the beta-adrenergic antagonist propranolol 10 minutes prior to cocaine injection (10 mg/kg, i.p.), and mice were tested in the EPM 20 minutes later. Shown is percent open arm time during the five minute EPM test. ** p<0.01 compared to vehicle control (N=10-17 per group).

FIG. 14 illustrates that the beta-adrenergic antagonist propranolol attenuates cocaine-induced anxiety in wild type C57BL6/J mice. Wild-type C57BL6/J mice were treated with either propranolol or saline 10 minutes prior to cocaine injection, and mice were tested on the EPM 20 minutes later. Shown is percent open time during the five minute EPM test. *p<0.05 compared to saline control (N=7 per group).

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### TERMS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated.

As used herein, the term “DBH” refers to the dopamine beta-hydroxylase protein, while “Dbh” is used to refer to the gene encoding the DBH protein.

As used herein “selective DBH inhibitor” refers to an inhibitor of the enzyme dopamine beta-hydroxylase (DBH) that does not substantially inhibit other proteins, enzymes, receptors and the like. For purposes of illustration, nepicastat is an example of a selective DBH inhibitor, while disulfuram, which inhibits a large class of enzymes (or proteins) including DBH, is an example of a non-selective DBH inhibitor. As used herein “DBH” refers to the dopamine 3-hydroxylase protein, while “Dbh” is used to refer to the dopamine 3-hydroxylase gene.

As used herein the term “stimulant addiction” or “stimulant dependence” refers to a condition wherein a host has an established habit of use of one or more stimulant drugs such as, but not limited to, cocaine, and amphetamines and derivatives thereof, such as methamphetamine, methylphenidate and the like.

The term “derivative” refers to a modification to the disclosed compounds.

As used herein the terms “treat,” “treating,” or “treatment” of a condition includes inhibiting the condition (slowing or arresting its development), relieving the condition (causing regression of the condition), and/or preventing recurrence or relapse of the condition. In the context of the present disclosure, the term may also refer to generating a physiological or psychological state that results in aversion to, and thereby, reduced acceptance of, a stimulant.

In certain embodiments, the disclosure contemplates preventing a condition that is treated herein.

The compounds of the present disclosure may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this disclosure may be generally prepared by reacting a free base with a suitable organic or inorganic acid. Representative salts of basic compounds of the present disclosure include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the disclosure carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

The disclosed compounds that contain an acidic moiety may form salts with a variety of organic and inorganic bases. Exemplary basic salts include ammonium salts; alkali metal salts such as sodium, lithium, and potassium salts; alkaline earth metal salts such as calcium and magnesium salts; salts with organic bases (for example, organic amines) such as benzathines, dicyclohexylamines, hydrabamines (formed with N,N-bis(dihydroabietyl)ethylenediamine), N-methyl-D-glucamines, N-methyl-D-glucamides, t-butyl amines; and salts with amino acids such as arginine, lysine, and the like.

Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Included are those esters and acyl groups known in the art for modifying the solubility or hydrolysis characteristics for use as sustained-release or prodrug formulations.

Solvates, and in particular, the hydrates of the compounds of the disclosure are included in the present disclosure as well.

The term “composition” as used herein is intended to encompass a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is preferred. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

The compounds of the present disclosure may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The pharmaceutical compositions for the administration of the compounds of this disclosure may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions may contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, flavoring, and coloring agents.

The pharmaceutical compositions may be in the form of a sterile injectable aqueous or oil-aqueous-suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The pharmaceutical composition and method of the present disclosure may further comprise other therapeutically active compounds as noted herein which are usually applied in the treatment of the above mentioned pathological conditions.

In the treatment or prevention of conditions that require selective inhibition of dopamine beta-hydroxylase enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing a stimulant addiction, such as a cocaine addiction, for which compounds of the present disclosure are indicated, generally satisfactory results are obtained when the compounds of the present disclosure are administered at a daily dosage of from about 0.1 mg to about 100 mg per kilogram of body weight, preferably given as a single daily dose or in divided doses two to six times a day, or in sustained release form. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. Advantageously, the present disclosure provides methods whereby the genotype of a patient in need of treatment with respect to the gene encoding dopamine beta-hydroxylase may be determined, and then correlated to the level of activity of the target enzyme. The dose of the administered therapeutic agent may then be adjusted to a level more appropriate to the patient based on whether the endogenous DBH level is high (homozygous wild-type), medium (heterozygous) or low (homozygous Dbh negative).

The term “therapeutically effective amount” as used herein refers to that amount of the compound being administered that will relieve to some extent one or more of the symptoms of the condition or disorder being treated. In reference to drug addiction (e.g., cocaine addiction), a therapeutically effective amount refers to that amount that has the effect, among others, of (1) causing the host to which it is administered to develop an aversion for the drug, and/or (2) reducing the amount of usage of the drug by the host, and/or (3) preventing a relapse of drug use in a previous user/addict.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples of excipients include, without limitation, calcium carbonate, calcium phosphate, various sugars and types of starch, cellulose derivatives, gelatin, vegetable oils, and polyethylene glycols.

To the extent that the disclosed compounds, and salts thereof, may exist in their tautomeric form, all such tautomeric forms are contemplated herein as part of the present disclosure.

All stereoisomers of the present compounds, such as those which may exist due to asymmetric carbons on the various substituents, including enantiomeric forms (which may exist even in the absence of asymmetric carbons) and diastereomeric forms, are contemplated within the scope of this disclosure. Individual stereoisomers of the compounds of the disclosure may, for example, be substantially free of other isomers, or may be admixed, for example, as racemates or with all other, or other selected, stereoisomers. The chiral centers of the compounds of the present disclosure can have the S or R configuration as defined by the IUPAC 1974 Recommendations.

As used herein, the term “genome” refers to all the genetic material in the chromosomes of a particular organism. Its size is generally given as its total number of base pairs. Within the genome, the term “gene” refers to an ordered sequence of nucleotides located in a particular position on a particular chromosome that encodes a specific functional product (e.g., a protein or RNA molecule). In general, a patient's genetic characteristics, as defined by the nucleotide sequence of its genome, are known as its “genotype,” while the patient's physical traits are described as its “phenotype.”

By “heterozygous” or “heterozygous polymorphism” is meant that the two alleles of a diploid cell or organism at a given locus are different, that is, that they have a different nucleotide exchanged for the same nucleotide at the same place in their sequences.

By “homozygous” or “homozygous polymorphism” is meant that the two alleles of a diploid cell or organism at a given locus are identical, that is, that they have the same nucleotide for nucleotide exchange at the same place in their sequences.

### DESCRIPTION

The present disclosure provides methods of treating drug addiction, specifically stimulant addiction (e.g., cocaine, amphetamine, methamphetamine, methylphenidate, etc.). Although it is not intended that the disclosure be limited by any particular mechanism, treatment is thought to work by modulating the activity of the catecholamine biosynthetic enzyme dopamine beta-hydroxylase (DBH) in the host. As described in greater detail below, the level of DBH activity in a host effects the host's physiological reaction to a stimulant drug, such as cocaine, which thereby effects the host's desire for the drug by altering the associations between the use of the drug and a particular physical state.

Studies involving the use of disulfuram to treat cocaine addicts indicated, as described below, that the mechanism of action of disulfuram with respect to cocaine use was different from the antabuse reaction that result in acetaldehyde accumulation, observed in alcoholics or others consuming alcohol. It was found that DBH inhibition was involved in disulfuram efficacy with cocaine users.

### DBH Inhibition and Drug Dependence.

Alcohol dependence and cocaine dependence show a remarkable degree of co-morbidity (Regier et al., (1990) JAMA 264:2511-2518; Carroll et al., (1993) J. Stud. Alcohol 54:199-208; Higgins et al., (1993) Am. J. Psychiatry 150:675-676). Preliminary findings supported the efficacy of disulfuram in cocaine/alcohol co-dependent individuals (Carroll et al., (1998) Addiction 93:713-727; Carroll et al., (2000) Addiction 95:1335-1349). However, the results from three studies strongly indicate that co-morbid alcohol use was not essential for disulfuram treatment of cocaine dependence, and in fact non-alcohol dependent subjects may benefit even more from disulfuram than those who also abuse alcohol (George et al., (2000) Biol. Psychiatry 47:1080-1086; Petrakis et al., (2000) Addiction 95:219-228; Carroll et al., (2004) Arch. Gen. Psychiatry 61:264-272). Because the drug combination of disulfuram and cocaine in the absence of alcohol does not result in acetaldehyde accumulation, the reduction of cocaine use by disulfuram had to depend on an interaction other than inhibition of aldehyde dehydrogenase. However, the mechanism for disulfuram efficacy was not clear.

Since the treatment of alcoholism with disulfuram appears to depend on its ability to create an aversive reaction to alcohol ingestion, a similar mechanism may have been responsible for its effect on cocaine dependence. This hypothesis is supported by those individuals receiving disulfuram treatment reporting a much higher incidence of paranoia associated with cocaine use than those not receiving disulfuram, suggesting that disulfuram increases the aversive properties of cocaine (Hameedi et al., (1995) Biol. Psychiatry 37:560-563; McCance-Katz et al., (1998) Drug Alcohol Depend.; 52(1):27-39; McCance-Katz et al., (1998) Biol. Psychiatry 43:540-3). Studies investigating the effects of disulfuram on responses to psychostimulants in animal models have shown that disulfuram decreases the locomotor response to acute administration of cocaine (Maj et al., (1968) J. Pharm. Pharmacol. 20:247-248) and amphetamine (Maj et al., (1968) J. Pharm. Pharmacol. 20:247-248.), enhances cocaine sensitization (Haile et al., (2003) Biol. Psychiatry 54:915-921), and attenuates the reinstatement phase of amphetamine self-administration in rodents (Davis et al., (1975) Pharmacol. Biochem. Behay. 3:477-484).

Cocaine-induced psychomotor activity and aversion is increased in dopamine beta-hydroxylase knockout (Dbh −/−) mice using the conditioned place preference paradigm. (Weinshenker et al., Proc. Natl. Acad. Sci. USA, 99: 13873-77, 2002; and Schank et al., Neuropsychopharmacology, 31:2221-30, 2006, both of which are hereby incorporated by reference in their entireties). Furthermore, cocaine-induced paranoia is increased in individuals either treated with disulfuram or with genetically low DBH activity (McCance-Katz et al., (1998) Drug Alcohol Depend. 52(1):27-39; McCance-Katz et al., (1998) Biol. Psychiatry 43:540-3). Taken together, these results suggest the existence of a second aversive “Antabuse reaction” that promotes cocaine abstinence, which is mediated by inhibition of DBH instead of aldehyde dehydrogenase.

Disulfuram inhibition of DBH and aldehyde dehydrogenase is similar, with IC₅₀ in the low micromolar range for both enzymes (Green A. L. (1964) Biochim. Biophys. Acta. 81:394-397.; Mays et al., (1998) Biochem. Pharmacol. 55:1099-103). Most inhibitors of DBH, including disulfuram, chelate copper, thereby depriving DBH of its required cofactor (Goldstein et al., (1964) Life Sci. 3:763-767). Disulfuram has been shown to inhibit DBH in animals, as evidenced by its ability to decrease norepinepherine (NE) and increase dopamine (DA) in peripheral and central tissues (Musacchio et al., (1966) J. Pharmacol. Exp. Ther. 152:56-61.; Karamanakos et al., (2001) Pharmacol. Toxicol. 88:106-110.; Bourdelat-Parks et al., (2005) Psychopharmacology 183:72-80). In humans, disulfuram decreases NE and its metabolites in urine, blood, and cerebrospinal fluid (Takahashi & Gjessing (1972) J. Psychiatr. Res. 9:293-314.; Major et al., (1979) Biol. Psychiatry 14:337-344; Rogers et al., (1979) Clin. Pharmacol. Ther. 25:469-477; Hoeldtke & Stetson (1980) J. Clin. Endocrinol. Metab. 51:810-815; Rosen & Lobo (1987) J. Clin. Endocrinol. Metab. 65:891-895; Paradisi et al., (1991) Acta Endocrinol. 125:246-252). Because the rewarding and aversive effects of cocaine are primarily mediated by NE and DA, it is contemplated that inhibition of DBH is important for the success of disulfuram treatment for cocaine dependence.

### Genetic Control of DBH Activity and Influence on Cocaine Addiction.

A proportion of the DBH protein is in the soluble fraction of NE secretory vesicles and is co-released upon stimulation of noradrenergic neurons. DBH activity can be readily measured in human serum and CSF. In serum, DBH activity and protein levels are strongly correlated and appear to represent the same biochemical phenotype. DBH activity is highly variable among individuals, and this variation has a strong (40-60%) genetic component (Weinshilboum R. M. (1979) Pharmacol. Rev. 30:133-166). A common polymorphism (allele frequency=0.22) in the promoter region of the human Dbh gene (a C to T change at nucleotide position −1021) was identified that accounts for much of the genetic variance in DBH activity (Zabetian et al., (2001) Am. J. Hum. Genet. 68:515-522). CT heterozygotes have about 50% DBH activity of that found in CC homozygotes, while TT homozygotes have very low DBH activity (about 10% of CC). There appears to be at least one other polymorphism, and perhaps more, that also contribute to variance in DBH activity, although with much smaller effects that −1021 (Tang et al., (2006) Biol Psychiatry 60:1034-1038).

“Low activity” Dbh alleles are significantly over-represented in addicts reporting cocaine-induced paranoia and significantly under-represented in those denying cocaine-induced paranoia (Cubells et al., (2000) Mol. Psychiatry. 5:56-63). Furthermore, individuals with low endogenous DBH activity are more susceptible to the aversive side effects of disulfuram, including psychosis (Heath et al., (1965) Dis. Nerv. Syst. 26:99-105; Ewing et al., (1977) Am. J. Psychiatry 134:927-928; Major et al., (1979) Biol. Psychiatry 14:337-344) and sedation (Ewing et al., (1978) Alcohol Clin. Exp. Res. 2:93-94). These observations support that some aversive properties of cocaine are enhanced as a result of genetic or pharmacological inhibition of DBH, and the effects of disulfuram on cocaine responses are influenced by a pharmacogenetic interaction between disulfuram and Dbh.

As demonstrated in the Examples below, disulfuram actually enhances cocaine sensitization, which is thought to model drug craving, and it has been argued that craving itself is aversive (Haile et al., (2003) Biol. Psychiatry 54:915-921). Therefore, the increased sensitivity to the psychomotor effects of psychostimulants in Dbh −/− mice suggests that a selective reduction of DBH activity may increase some of the aversive properties of psychostimulants. These results suggest that increased cocaine aversion due to DBH inhibition contributes to disulfuram-induced cocaine abstinence. Therefore, as demonstrated in Example 3 below, and FIGS. 8-10, selective DBH inhibitors can function as effective pharmacotherapy for psychostimulant dependence.

Another possible NE-related mechanism of disulfuram efficacy would be the prevention of a relapse. It was first demonstrated more than 25 years ago that both disulfuram and U-14,624, another DBH inhibitor, block reinstatement of amphetamine self-administration in rats (Davis et al., (1975) Pharmacol. Biochem. Behay. 3:477-484). Additional studies have demonstrated that drugs that attenuate NE release or signaling also block footshock-induced reinstatement of cocaine self-administration, which is thought to model stress-induced relapse (Erb et al., (2000) Neuropsychopharmacology 23:138-150; Shaham et al., (2000) Brain Res. Brain Res. Rev. 33(1):13-33; Leri et al., (2002) J. Neurosci. 22:5713-5718). Administration of 2-adrenergic antagonists, which increase NE release by blocking autoreceptors, can reinstate cocaine seeking behavior is squirrel monkeys (Lee et al., (2004) Neuropsychopharmacology 29:686-693). NE has been implicated in stress responses, and the “stress” peptide corticotropin-releasing factor (CRF) is also critical for footshock-induced cocaine reinstatement (Erb et al., (1998) J. Neurosci. 18:5529-5536; Koob et al., (1998) Neuron 21: 467-476). Furthermore, the effects of the NE and CRF pathways on cocaine reinstatement show a remarkable degree of localized interaction (Erb & Stewart (1999) J. Neurosci. 19:RC35; Erb et al., (2001) Psychopharmacology (Berl) 158:360-365; Leri et al., (2002) J. Neurosci. 22:5713-5718). These results indicate that the noradrenergic system may be involved in stress-induced relapse. NE signaling via alpha-1 adrenergic receptors has recently been shown to be required for cocaine-primed reinstatement of cocaine seeking in rats (Zhang & Kosten (2005) Biol Psychiatry 57:1202-1204). The effects of NE system manipulations on cue-induced reinstatement of drug seeking have not been investigated, but it may be important given the canonical role of NE neurons in attention and arousal to external stimuli. It is contemplated, therefore, that selective DBH inhibition could interfere with the processes underlying relapse and promote abstinence in cocaine-dependent individuals. In addition, because other abused stimulant drugs (e.g., amphetamine derivatives) utilize similar neurocircuitry for their addictive potential, selective DBH inhibition is also likely to be effective in treating individuals with dependence on other psychostimulants.

Methods of Treatment

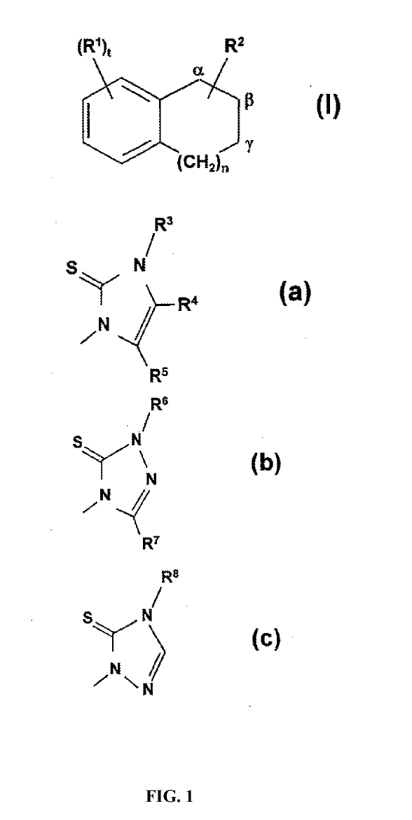

(a) Compositions for treating stimulant addiction: Specifically, the present disclosure provides methods that include inhibiting the activity of dopamine 3-hydroxylase (DBH) in a host with a stimulant (e.g., cocaine) addiction by administering to the host a therapeutically effective amount of a selective DBH inhibitor. While the methods of the present disclosure may be practiced with any pharmaceutically acceptable selective DBH inhibitor, some non-limiting examples include, but are not limited to, benzocycloalkylazolethione derivatives, such as those structures illustrated in FIGS. 1-4, and described in detail, including methods of manufacture thereof, in U.S. Pat. Nos. 5,719,280; 5,438,150; and 5,538,988, which are hereby incorporated by reference in their entirety.

Advantageous compounds for use in the methods of the present disclosure include, but are not limited to, a compound of Formula I (as shown in FIG. 1), in which: n is 0, 1 or 2; t is 0, 1, 2 or 3; R¹ is independently halo, hydroxy or (C₁₋₄) alkyloxy; and R² is attached at the alpha-, beta- or gamma-position and is a group selected from the Formulae (a), (b) and (c): shown in FIG. 1, in which: R⁴ is hydro, R³ is hydro or —(CH₂)_{q}R⁹ {in which q is 0, 1, 2, 3 or 4 and R⁹ is carboxy, (C₁₋₄) alkyloxycarbonyl, carbamoyl or a group selected from aryl and heteroaryl (which group is optionally further substituted with one to two substituents independently selected from hydroxy, (C₁₋₄) alkyloxy, cyano, 1H-tetrazol-5-yl, carboxy and (C₁₋₄) alkyloxycarbonyl)} and R⁵ is hydro or —NHR¹⁰ {in which R¹⁵ is hydro, (C₁₋₄) alkanoyl, trifluoro(C₁₋₄) alkanoyl, carbamoyl, (C₁₋₄) alkyloxycarbonyl, (C₁₋₄) alkylcarbamoyl, di(C₁₋₄) alkylcarbamoyl, amino (C₁₋₄) alkanoyl, (C₁₋₄) alkylamino (C₁₋₄) alkanoyl, di(C₁₋₄) alkylamino (C₁₋₄) alkanoyl, a group selected from aroyl and heteroaroyl (which aroyl and heteroaroyl are optionally further substituted with one to two substituents independently selected from hydroxy, (C₁₋₄) alkyloxy, cyano, 1H-tetrazol-5-yl, carboxy and (C₁₋₄) alkyloxycarbonyl) or —C(NR¹¹)NHR¹² (in which R¹¹ and R¹² are independently hydro, acetyl or tert-butoxycarbonyl)}; or R⁴ and R⁵ are each hydro and R³ is —NHR¹⁶ (in which R¹⁹ is as defined above); or R⁵ is hydro, R³ is hydro or —(CH₂)_{c}, R⁹ (in which q and R⁹ are as defined above) and R⁴ is (C₁₋₄) alkyl, di(C₁₋₄) alkylaminomethyl, piperidin-1-ylmethyl, morpholin-4-ylmethyl, formyl, 1-hydroxy (C₁₋₄) alkyl or —CH₂NHR¹³ {in which R¹³ is hydro, (C₁₋₄alkyl, (C₁₋₄alkanoyl, trifluoro (C₁₋₄alkanoyl, carbamoyl, (C₁₋₄alkyloxycarbonyl, (C₁₋₄alkylcarbamoyl, di(C₁₋₄) alkylcarbamoyl, amino (C₁₋₄) alkanoyl, (C₁₋₄) alkylamino (C₁₋₄) alkanoyl, di(C₁₋₄) alkylamino (C₁₋₄alkanoyl, carboxy (C₁₋₄alkyl, (C₁₋₄) alkyloxycarbonyl (C₁₋₄alkyl, carbamoyl (C₁₋₄alkyl, a group selected from aroyl, heteroaroyl, aryl (C₁₋₄alkyl and heteroaryl (C₁₋₄alkyl (which aroyl, heteroaroyl, aryl and heteroaryl are optionally further substituted with one to two substituents independently selected from hydroxy, (C₁₋₄alkyloxy, cyano, 1H-tetrazol-5-yl, carboxy and (C₁₋₄alkyloxycarbonyl) or —C(NR¹¹)NHR¹² (in which R¹¹ and R¹² are as defined above)}; or R³ is hydro or —(CH²)_{q}R⁹ (in which q and R⁹ are as defined above), R⁴ is hydro, (C₁₋₄) alkyl or —C(O)R¹⁴ (in which R¹⁴ is amino, hydroxy (C₁₋₄) alkyloxy, 2-(dimethylamino)ethylamino, 4-methylpiperazin-1-yl, 2-(dimethylamino)ethylmercapto, 4-(methylsulfonylamino) anilino or 1H-tetrazol-5-ylamino) and R⁵ is cyano, hydroxymethyl, 1H-tetrazol-5-yl, 4,5-dihydroimidazol-2-yl, pyrrolidin-1-ylmethyl, piperidin-1-ylmethyl, morpholin-4-ylmethyl, piperazin-1-ylmethyl, 4-(C₁₋₄) alkylpiperazin-1-ylmethyl, —C(O)R¹⁴ (in which R¹⁴ are as defined above), —C(NH)NR¹⁵R¹⁶ (in which R¹⁵ and R¹⁶ independently hydro, (C₁₋₄alkyl or trifluoro(C₁₋₄ alkyl) or —CH₂ NR¹⁶R¹⁷ (in which R¹⁹ is as defined above and R¹⁷ is hydro or C₁₋₄alkyl); or R³ is hydro or —(CH₂)_{q}R⁹ (in which q and R⁹ are as defined above) and R⁴ and R⁵ are dependently di(C₁₋₄) alkylaminomethyl, piperidin-1-ylmethyl, morpholin-4-ylmethyl or hydroxymethyl; R⁶ is hydro, 2-carboxyethyl, 2-carbamoylethyl or 2-(C₁₋₄) alkyloxycarbonylethyl; R⁷ is hydro, pyrrolidin-1-ylmethyl, piperidin-1-ylmethyl, morpholin-4-ylmethyl, piperazin-1-ylmethyl, 4-(C₁₋₄) alkylpiperazin-1-ylmethyl or —CH₂NR¹⁶R¹⁷ (in which R¹⁹ and R¹⁷ are as defined above); and R⁸ is hydro, 2-carboxyethyl, 2-carbamoylethyl, 2-(C₁₋₄) alkyloxycarbonylethyl or —NHR¹⁹ (in which R¹⁰ are as defined above); and the pharmaceutically acceptable salts, individual isomers, and mixtures of isomers thereof.

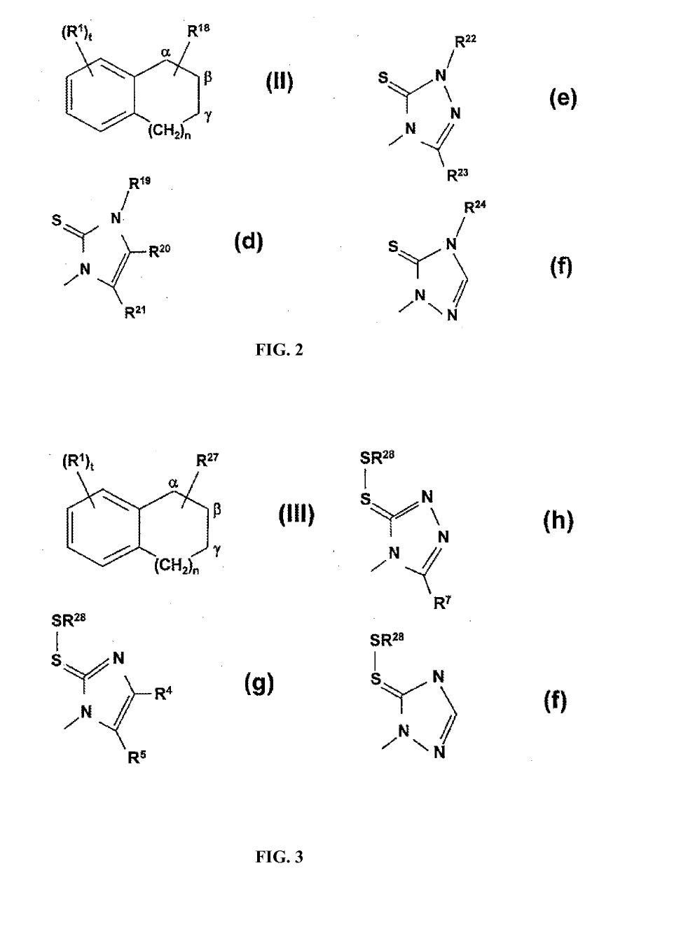

Another advantageous compound useful in the methods of the present disclosure is a compound of Formula II as illustrated in FIG. 2, in which: n is 0, 1 or 2; t is 0, 1, 2 or 3; R¹ is independently halo, hydroxy or (C₁₋₄ alkyloxy; and R¹⁸ is attached at the alpha-, beta- or gamma-position and is a group selected from Formulae (d), (e) and (f) as shown in FIG. 2, in which: R²⁰ is hydro, R¹⁹ is hydro or —(CH₂)_{q}R⁹ {in which q is 0, 1, 2, 3 or 4 and R⁹ is carboxy, (C₁₋₄) alkyloxycarbonyl, carbamoyl or a group selected from aryl and heteroaryl (which group is optionally further substituted with one to two substituents independently selected from hydroxy, (C₁₋₄) alkyloxy, cyano, 1H-tetrazol-5-yl, carboxy and (C₁₋₄) alkyloxycarbonyl)} and R²¹ is —NR²⁵R²⁶ (in which R²⁵ is hydro or (C₁₋₄) alkyl and R²⁶ is L-alanyl, L-arginyl, L-asparaginyl, L-.alpha.-aspartyl, L-beta-aspartyl, L-cysteinyl, L-glutaminyl, L-alpha-glutamyl, L-.gamma.-glutamyl, N—(C₁₋₄) alkanoyl-L-alpha-glutamyl, N—(C₁₋₄) alkanoyl-L-.gamma.-glutamyl, glycyl, L-histidyl, L-isoleucyl, L-leucyl, L-lysyl, L-methionyl, L-ornithinyl, L-phenylalanyl, L-prolyl, L-seryl, L-threonyl, L-tryptophyl, L-tyrosyl, L-valyl, 1-amino-cyclopropylcarbonyl, 1-aminocyclobutylcarbonyl, 1-aminocyclopentylcarbonyl or 1-aminocyclohexylcarbonyl); or R²⁰ and R²¹ are each hydro and R¹⁹ is —NR²⁵R²⁶ (in which R²⁵ and R²⁶ are as defined above); or R²¹ is hydro, R¹⁹ is hydro or —(CH₂)_{q}R⁹ (in which q and R⁹ are as defined above) and R²⁰ is —CH₂NR²⁵R²⁶ (in which R²⁵ and R²⁶ are as defined above); or R¹⁹ is hydro or —(CH₂)_{q}R⁹ (in which q and R⁹ are as defined above), R²⁰ is hydro, (C₁₋₄) alkyl or —C(O)R¹⁴ (in which R¹⁴ is amino, hydroxy (C₁₋₄) alkyloxy, 2-(dimethylamino)ethylamino, 4-methylpiperazin-1-yl, 2-(dimethylamino)ethylmercapto, 4-(methylsulfonylamino) anilino or 1H-tetrazol-5-ylamino) and R²¹ is —CH₂NR²⁵R²⁶ (in which R²⁵ and R²⁶ is as defined above); and R²² is hydro, 2-carboxyethyl, 2-carbamoylethyl or 2-(C₁₋₄) alkyloxycarbonylethyl; R²³ is —CH₂NR²⁵R²⁶(in which R²⁵ and R²⁶ are as defined above); and R²⁴ is —NR²⁵R²⁶ (in which R²⁵ and R²⁶ are as defined above); and the pharmaceutically acceptable salts, individual isomers, and mixtures of isomers thereof.

Another advantageous compound of use in the methods and compositions of the disclosure is the compound of Formula III as shown in FIG. 3, in which: n is 0, 1 or 2; t is 0, 1, 2 or 3; R¹ is independently halo, hydroxy or (C₁₋₄) alkyloxy; and R²⁷ is attached at the alpha-, beta- or gamma-position and is a group selected from Formulae (g), (h) and (i) shown in FIG. 3, in which: R⁴ is hydro and R⁵ is hydro or —NHR¹⁰ {in which R¹⁰ is hydro, (C₁₋₄) alkanoyl, trifluoro(C₁₋₄) alkanoyl, carbamoyl, (C₁₋₄) alkyloxycarbonyl, (C₁₋₄) alkylcarbamoyl, di(C₁₋₄) alkylcarbamoyl, amino (C₁₋₄) alkanoyl, (C₁₋₄) alkylamino (C₁₋₄) alkanoyl, di(C₁₋₄) alkylamino (C₁₋₄) alkanoyl, a group selected from aroyl and heteroaroyl (which aroyl and heteroaroyl are optionally further substituted with one to two substituents independently selected from hydroxy, (C₁₋₄) alkyloxy, cyano, 1H-tetrazol-5-yl, carboxy and (C₁₋₄) alkyloxycarbonyl) or —C(NR¹¹)NHR¹² (in which R¹¹ and R¹² are independently hydro, acetyl or tert-butoxycarbonyl)}; or R⁵ is hydro and R⁴ is (C₁₋₄) alkyl, di(C₁₋₄) alkylaminomethyl, piperidin-1-ylmethyl, morpholin-4-ylmethyl, 1-hydroxy(C₁₋₄ alkyl or —CH₂NHR¹³ {in which R¹³ is hydro, (C₁₋₄ alkyl, (C₁₋₄) alkanoyl, trifluoro(C₁₋₄) alkanoyl, carbamoyl, (C₁₋₄) alkyloxycarbonyl, (C₁₋₄) alkylcarbamoyl, di(C₁₋₄) alkylcarbamoyl, amino (C₁₋₄) alkanoyl, (C₁₋₄) alkylamino (C₁₋₄) alkanoyl, di(C₁₋₄ alkylamino (C₁₋₄ alkanoyl, carboxy (C₁₋₄) alkyl, (C₁₋₄ alkyloxycarbonyl (C₁₋₄ alkyl, carbamoyl (C₁₋₄) alkyl, a group selected from aroyl, heteroaroyl, aryl (C₁₋₄ alkyl and heteroaryl (C₁₋₄) alkyl (which aroyl, heteroaroyl, aryl and heteroaryl are optionally further substituted with one to two substituents independently selected from hydroxy, (C₁₋₄) alkyloxy, cyano, 1H-tetrazol-5-yl, carboxy and (C₁₋₄) alkyloxycarbonyl) or —C(NR¹¹)NHR¹² (in which R¹¹ and R¹² are as defined above)}; or R⁴ is hydro, (C₁₋₄) alkyl or —C(O)R¹⁴ (in which R¹⁴ is amino, hydroxy (C₁₋₄) alkyloxy, 2-(dimethylamino)ethylamino, 4-methylpiperazin-1-yl, 2-(dimethylamino)ethylmercapto, 4-(methylsulfonylamino) anilino or 1H-tetrazol-5-ylamino) and R⁵ is hydroxymethyl, 1H-tetrazol-5-yl, 4,5-dihydroimidazol-2-yl, pyrrolidin-1-ylmethyl, piperidin-1-ylmethyl, morpholin-4-ylmethyl, piperazin-1-ylmethyl, 4-(C₁₋₄) alkylpiperazin-1-ylmethyl, —C(O)R¹⁴ (in which R¹⁴ are as defined above), —C(NH)NR¹⁵R¹⁶ (in which R¹⁵ and R¹⁶ are independently hydro, (C₁₋₄) alkyl or trifluoro(C₁₋₄ alkyl) or —CH₂NR¹⁰R¹⁷ (in which R¹⁰ is as defined above and R¹⁷ is hydro or C₁₋₄) alkyl); or R⁴ and R⁵ are dependently di(C₁₋₄) alkylaminomethyl, piperidin-1-ylmethyl, morpholin-4-ylmethyl or hydroxymethyl; R⁶ is hydro, 2-carboxyethyl, 2-carbamoylethyl or 2-(C₁₋₄) alkyloxycarbonylethyl; R⁷ is hydro, pyrrolidin-1-ylmethyl, piperidin-1-ylmethyl, morpholin-4-ylmethyl, piperazin-1-ylmethyl, 4-(C₁₋₄) alkylpiperazin-1-ylmethyl or —CH₂NR¹⁰R¹⁷ and R¹⁷ are as defined above); and R²⁸ is (C₂₋₆) alkyl {which alkyl is further substituted by one to two substituents independently selected from —N(R²⁹)₂, —C(O)OR³⁰, —PO(OR³⁰)₂, —SO₃R³⁰, —SO₂NHR³⁰ and —OR³⁰ (in which each R²⁹ is independently hydro, acetyl or trifluoroacetyl and each R³⁰ is independently hydro or (C₁₋₆) alkyl)}; and the pharmaceutically acceptable salts, individual isomers, and mixtures of isomers thereof.

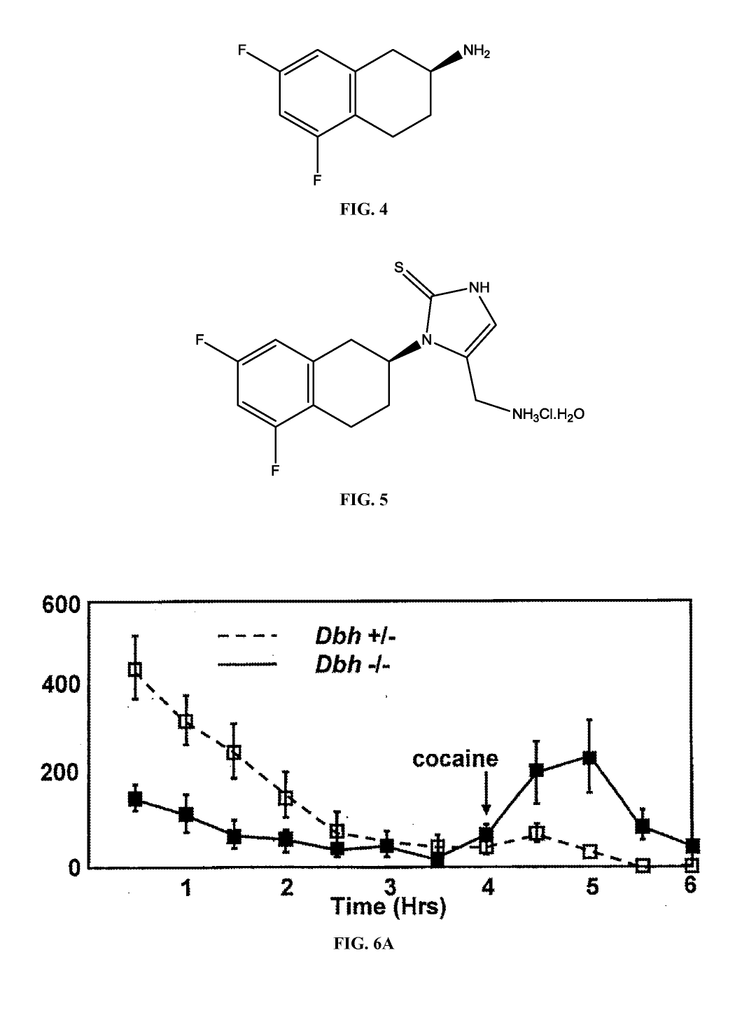

One advantageous compound is (S)-5,7,-difluoro-1,2,3,4-tetrahydronapthalen-2-ylamine as shown in FIG. 4. Most advantageous for use in the methods and compositions of the present disclosure is the selective DBH inhibitor Nepicastat (S-5-aminomethyl-1-(5,7-difluoro-1,2,3,4-tetrahydronaphth-2-yl)-1,3-dihydroimidazole-2-thione hydrochloride) as shown in FIG. 5.

The methods of the present disclosure also include determining an appropriate therapeutically effective amount of a selective DBH inhibitor suitable for a particular host by determining the natural activity level of DBH in the host. Because DBH activity in humans is genetically controlled, the DBH genotype will be an important determinant of treatment efficacy. For example, individuals with genetically low DBH activity will require a lower dose of selective DBH inhibitor than individuals with genetically high DBH activity.

In other words, since a host with a naturally low level of DBH will be likely to have greater sensitivity to the effects of a drug such as cocaine, as discussed above, such a host would likely need a lower dose of a selective DBH inhibitor compound than a host with a normal or high genetic DBH level.

It will be understood, however, that the total daily usage of the compositions of the present disclosure will be decided by the attending physician within the scope of sound medical judgment. In addition to the genetic DBH level of a host, the specific therapeutically effective dose level for any particular host can depend upon a variety of factors, including, but not limited to, the addiction or other condition being treated and the severity of the addiction; the activity of the specific composition employed; the specific composition employed; the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the composition at levels lower than those required to achieve the desired therapeutic effect and to gradually increase the dosage until the desired effect is achieved.

The selective DBH inhibitors may be administered to a host in need thereof in any number of pharmaceutically acceptable dosage forms. Typically, the selective DBH inhibitor compound or a pharmaceutically acceptable salt thereof will be combined with a pharmaceutically acceptable carrier and/or excipient. Other additives known to those of skill in the art may also be included in the pharmaceutically acceptable composition, depending on the dosage form used, such as stabilizers, emulsifiers, solubilizers, binders, fillers, disintegrants, lubricants, penetration enhancers, preservatives, and the like. Various dosage forms may be used depending on the mode of administration to be used.

A pharmaceutically acceptable composition including a selective DBH inhibitor can be administered via routes such as, but not limited to, topical treatments (e.g., cream, gel, patch, sprays, and the like), transdermal patches, IV, IM, and the like. In a particular, embodiments of the present disclosure can be injected into a localized area using a syringe or like device, or delivered orally.

Pharmaceutical compositions and dosage forms of the disclosure include a pharmaceutically acceptable salt of the compound and/or a pharmaceutically acceptable polymorph, solvate, hydrate, dehydrate, co-crystal, anhydrous, or amorphous form thereof. Specific salts of disclosed compounds include, but are not limited to, sodium, lithium, and potassium salts, and hydrates thereof.

Pharmaceutical unit dosage forms of the selective DBH inhibitor compounds of this disclosure are suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure typically vary depending on their use. For example, a dosage form used in the acute treatment of a condition or disorder may contain larger amounts of the active ingredient, e.g., the disclosed compounds or combinations thereof, than a dosage form used in the chronic treatment of the same condition or disorder. Similarly, a parenteral dosage form may contain smaller amounts of the active ingredient than an oral dosage form used to treat the same condition or disorder. These and other ways in which specific dosage forms encompassed by this disclosure vary from one another will be readily apparent to those skilled in the art (See, e.g., Remington's Pharmaceutical Sciences, 18th ed., Mack Publishing, Easton, Pa. (1990)).

### Determination of Patient Genotype and Determination of Effective Therapeutic Agent Dose

To determine the genotype of a patient according to the methods of the present disclosure, it is necessary to obtain a sample of genomic DNA from that patient. Typically, that sample of genomic DNA will be obtained from a sample of tissue or cells taken from that patient.

A tissue or cell sample may be taken from a patient at any time in the lifetime of the patient for the determination of a germline polymorphism. The tissue sample can comprise hair (including roots), buccal swabs, blood, saliva, semen, muscle, or from any internal organs. In the methods of the present disclosure, the source of the tissue sample, and thus also the source of the test nucleic acid sample, is not critical. For example, the test nucleic acid can be obtained from cells within a body fluid of the patient, or from cells constituting a body tissue of the patient. The particular body fluid from which cells are obtained is also not critical to the present disclosure. For example, the body fluid may be selected from the group consisting of blood, ascites, pleural fluid, and spinal fluid. Furthermore, the particular body tissue from which cells are obtained is also not critical to the present disclosure. For example, the body tissue can include, but is not limited to, skin, endometrial, uterine, and cervical tissue. Whatever source of cells or tissue is used, a sufficient amount of cells must be obtained to provide a sufficient amount of DNA for analysis. This amount will be known or readily determinable by those skilled in the art.

DNA is isolated from the tissue/cells by techniques known to those skilled in the art (see, e.g., U.S. Pat. Nos. 6,548,256 and 5,989,431, Hirota et al., (1989) Jinrui Idengaku Zasshi. 34(3):217-23 and John et al., (1991) Nucleic Acids Res. 25; 408; the disclosures of which are incorporated by reference in their entireties). For example, high molecular weight DNA may be purified from cells or tissue using proteinase K extraction and ethanol precipitation. DNA may be extracted from a patient specimen using any other suitable methods known in the art.

It is an object of the present disclosure to determine the genotype of a given patient to identify patients carrying specific alleles of the Dbh locus, and in particular a CT transition (as determined by Zabetian et al., (2001) Am. J. Hum. Genet. 68:515-522) compared to a control sequence. There are many methods known in the art for determining the genotype of an patient and for identifying whether a given DNA sample contains a particular polymorphism. Any method for determining genotype can be used for determining the genotype in the present disclosure. Such methods include, but are not limited to, amplimer sequencing, DNA sequencing, fluorescence spectroscopy, fluorescence resonance energy transfer (or “FRET”)-based hybridization analysis, high throughput screening, mass spectroscopy, nucleic acid hybridization, polymerase chain reaction (PCR), RFLP analysis and size chromatography (e.g., capillary or gel chromatography), all of which are well known to one of skill in the art. In particular, methods for determining nucleotide polymorphisms, particularly single nucleotide polymorphisms, are described in U.S. Pat. Nos. 6,514,700; 6,503,710; 6,468,742; 6,448,407; 6,410,231; 6,383,756; 6,358,679; 6,322,980; 6,316,230; and 6,287,766 and reviewed by Chen & Sullivan, (2003) Pharmacogenomics J.; 3(2):77-96, the disclosures of which are incorporated by reference in their entireties.

In one embodiment, the presence or absence of the CT transition of the Dbh locus is determined by sequencing the region of the genomic DNA sample that spans the polymorphic locus. Many methods of sequencing genomic DNA are known in the art, and any such method can be used, see for example Sambrook et al., Molecular Cloning; A Laboratory Manual 2d ed. (1989). For example, as described below, a DNA fragment spanning the location of the polymorphism of interest can amplified using the polymerase chain reaction or some other cyclic polymerase mediated amplification reaction. The amplified region of DNA can then be sequenced using any method known in the art. Advantageously, the nucleic acid sequencing is by automated methods (reviewed by Meldrum, (2000) Genome Res. 10:1288-303, the disclosure of which is incorporated by reference in its entirety), for example using a Beckman CEQ 8000 Genetic Analysis System (Beckman Coulter Instruments, Inc.). Methods for sequencing nucleic acids include, but are not limited to, automated fluorescent DNA sequencing (see, e.g., Watts & MacBeath, (2001) Methods Mol. Biol.; 167:153-70 and MacBeath et al., (2001) Methods Mol. Biol.; 167:119-52), capillary electrophoresis (see, e.g., Bosserhoff et al., (2000) Comb. Chem. High Throughput Screen. 3:455-66), DNA sequencing chips (see, e.g., Jain, (2000) Pharmacogenomics. 1:289-307), mass spectrometry (see, e.g., Yates, (2000) Trends Genet. 16(1):5-8), pyrosequencing (see, e.g., Ronaghi, (2001) Genome Res. 11:3-11), and ultrathin-layer gel electrophoresis (see, e.g., Guttman & Ronai, (2000) Electrophoresis. 21:3952-64), the disclosures of which are hereby incorporated by reference in their entireties. The sequencing can also be done by any commercial company. Examples of such companies include, but are not limited to, the University of Georgia Molecular Genetics Instrumentation Facility (Athens, Ga.) or SeqWright DNA Technologies Services (Houston, Tex.).

The detection of a given SNP can be performed using cyclic polymerase-mediated amplification methods. Any one of the methods known in the art for amplification of DNA may be used, such as for example, the polymerase chain reaction (PCR), the ligase chain reaction (LCR) (Barany F., (1991) Proc. Natl. Acad. Sci. USA 88:189-193), the strand displacement assay (SDA), or the oligonucleotide ligation assay (“OLA”) (Landegren et al., (1988) Science 241:1077-1080). Nickerson et al. have described a nucleic acid detection assay that combines attributes of PCR and OLA (Nickerson et al., (1990) Proc. Natl. Acad. Sci. USA 87:8923-8927). Other known nucleic acid amplification procedures, such as transcription-based amplification systems (Malek et al., U.S. Pat. No. 5,130,238; Davey et al., European Patent Application 329,822; Schuster et al., U.S. Pat. No. 5,169,766; Miller et al., PCT Application WO89/06700; Kwoh et al., (1989) Proc. Natl. Acad. Sci. USA 86:1173; Gingeras et al., PCT Application WO88/10315)), or isothermal amplification methods (Walker et al., (1992) Proc. Natl. Acad. Sci. USA 89:392-396) may also be used.

The most advantageous method of amplifying DNA fragments containing the SNPs of the disclosure employs PCR (see e.g., U.S. Pat. Nos. 4,965,188; 5,066,584; 5,338,671; 5,348,853; 5,364,790; 5,374,553; 5,403,707; 5,405,774; 5,418,149; 5,451,512; 5,470,724; 5,487,993; 5,523,225; 5,527,510; 5,567,583; 5,567,809; 5,587,287; 5,597,910; 5,602,011; 5,622,820; 5,658,764; 5,674,679; 5,674,738; 5,681,741; 5,702,901; 5,710,381; 5,733,751; 5,741,640; 5,741,676; 5,753,467; 5,756,285; 5,776,686; 5,811,295; 5,817,797; 5,827,657; 5,869,249; 5,935,522; 6,001,645; 6,015,534; 6,015,666; 6,033,854; 6,043,028; 6,077,664; 6,090,553; 6,168,918; 6,174,668; 6,174,670; 6,200,747; 6,225,093; 6,232,079; 6,261,431; 6,287,769; 6,306,593; 6,440,668; 6,468,743; 6,485,909; 6,511,805; 6,544,782; 6,566,067; 6,569,627; 6,613,560; 6,613,560 and 6,632,645; the disclosures of which are incorporated by reference in their entireties), using primer pairs that are capable of hybridizing to the proximal sequences that define or flank a polymorphic site in its double-stranded form.

To perform a cyclic polymerase mediated amplification reaction according to the present disclosure, the primers are hybridized or annealed to opposite strands of the target DNA, the temperature is then raised to permit the thermostable DNA polymerase to extend the primers and thus replicate the specific segment of DNA spanning the region between the two primers. Then the reaction is thermocycled so that at each cycle the amount of DNA representing the sequences between the two primers is doubled, and specific amplification of the ob gene DNA sequences, if present, results.

Any of a variety of polymerases can be used in the present disclosure. For thermocyclic reactions, the polymerases are thermostable polymerases such as Taq, KlenTaq, Stoffel Fragment, Deep Vent, Tth, Pfu, Vent, and UITma, each of which are readily available from commercial sources. For non-thermocyclic reactions, and in certain thermocyclic reactions, the polymerase will often be one of many polymerases commonly used in the field, and commercially available, such as DNA pol 1, Klenow fragment, T7 DNA polymerase, and T4 DNA polymerase. Guidance for the use of such polymerases can readily be found in product literature and in general molecular biology guides.

Typically, the annealing of the primers to the target DNA sequence is carried out for about 2 minutes at about 37-55 degree C., extension of the primer sequence by the polymerase enzyme (such as Taq polymerase) in the presence of nucleoside triphosphates is carried out for about 3 minutes at about 70-75 degree C., and the denaturing step to release the extended primer is carried out for about 1 minute at about 90-95 degree C. However, these parameters can be varied, and one of skill in the art would readily know how to adjust the temperature and time parameters of the reaction to achieve the desired results. For example, cycles may be as short as 10, 8, 6, 5, 4.5, 4, 2, 1, 0.5 minutes or less.

Also, “two temperature” techniques can be used where the annealing and extension steps may both be carried out at the same temperature, typically between about 60-65 degree C., thus reducing the length of each amplification cycle and resulting in a shorter assay time.

Typically, the reactions described herein are repeated until a detectable amount of product is generated. Often, such detectable amounts of product are between about 10 ng and about 100 ng, although larger quantities, e.g. 200 ng, 500 ng, 1 .mu.g or more can also, of course, be detected. In terms of concentration, the amount of detectable product can be from about 0.01 pmol, 0.1 pmol, 1 pmol, 10 pmol, or more. Thus, the number of cycles of the reaction that are performed can be varied, the more cycles are performed, the more amplified product is produced. In certain embodiments, the reaction comprises 2, 5, 10, 15, 20, 30, 40, 50, or more cycles.

For example, the PCR reaction may be carried out using about 25-50 μl samples containing about 0.01 to 1.0 ng of template amplification sequence, about 10 to 100 pmol of each generic primer, about 1.5 units of Taq DNA polymerase (Promega Corp.), about 0.2 mM dDATP, about 0.2 mM dCTP, about 0.2 mM dGTP, about 0.2 mM dTTP, about 15 mM MgCl₂, about 10 mM Tris-HCl (pH 9.0), about 50 mM KCl, about 1 μg/ml gelatin, and about 10 μl/ml Triton X-100.

Those of skill in the art are aware of the variety of nucleotides available for use in the cyclic polymerase mediated reactions. Typically, the nucleotides can be at least in part of deoxynucleotide triphosphates (dNTPs), which are readily commercially available. Parameters for optimal use of dNTPs are also known to those of skill, and are described in the literature. In addition, a large number of nucleotide derivatives are known to those of skill and can be used in the present reaction. Such derivatives include fluorescently labeled nucleotides, allowing the detection of the product including such labeled nucleotides, as described below. Also included in this group are nucleotides that allow the sequencing of nucleic acids including such nucleotides, such as chain-terminating nucleotides, dideoxynucleotides and boronated nuclease-resistant nucleotides. Commercial kits containing the reagents most typically used for these methods of DNA sequencing are available and widely used. Other nucleotide analogs include nucleotides with bromo-, iodo-, or other modifying groups, which affect numerous properties of resulting nucleic acids including their antigenicity, their replicatability, their melting temperatures, their binding properties, etc. In addition, certain nucleotides include reactive side groups, such as sulfhydryl groups, amino groups, N-hydroxysuccinimidyl groups, that allow the further modification of nucleic acids comprising them.

Primers for the detection of polymorphisms in the Dbh locus can be oligonucleotide fragments. Such fragments should be of sufficient length to enable specific annealing or hybridization to the nucleic acid sample. The sequences typically will be about 8 to about 44 nucleotides in length, but may be longer. Longer sequences, e.g., from about 14 to about 50, are advantageous for certain embodiments.

In embodiments where it is desired to amplify a fragment of DNA comprising the U50 locus, primers having contiguous stretches of about 8, 9, 10, 11, 12, 13, 14, 15, 16, 17, 18, 19, 20, 21, 22, 23, or 24 nucleotides from the genomic sequence encompassing the Dbh locus (GenBank Accession Nos: AC000404 and AC001227) are contemplated.

Although various different lengths of primers can be used, and the exact location of the stretch of contiguous nucleotides in Dbh gene used to make the primer can vary, it is important that the sequences to which the forward and reverse primers anneal are located on either side of the particular nucleotide positions that may be polymorphic variants of the Dbh locus. For example, when designing primers for amplification of the CT polymorphism of Dbh (Zabetian et al., (2001) Am. J. Hum. Genet. 68:515-522 incorporated herein by reference in its entirety), one primer must be located upstream of (but not overlapping with) nucleotide position −1021 of the promoter region of the Dbh gene (Zabetian et al., (2001) Am. J. Hum. Genet. 68:515-522), and the other primer must be located downstream of (but not overlapping with) nucleotide position −1021 of the promoter region of the Dbh gene (Zabetian et al., (2001) Am. J. Hum. Genet. 68:515-522).

The above methods employ primers located on either side of, and not overlapping with the nucleotide position −1021 of the promoter region of the Dbh gene to amplify a fragment of DNA that includes the nucleotide position at which the polymorphism is located. Such methods require additional steps, such as sequencing of the fragment, or hybridization of allele specific probes to the fragment, in order to determine the genotype at the polymorphic site. However, in some embodiments of the present disclosure, the amplification method is itself a method for determining the genotype of the polymorphic site, as for example, in “allele-specific PCR”. In allele-specific PCR, primer pairs are chosen such that amplification itself is dependent upon the input template nucleic acid containing the polymorphism of interest. In such embodiments, primer pairs are chosen such that at least one primer spans the actual nucleotide position of the polymorphism and is therefore an allele-specific oligonucleotide primer. Typically, a primer contains a single allele-specific nucleotide at the 3′ terminus preceded by bases that are complementary to the gene of interest. The PCR reaction conditions are adjusted such that amplification by a DNA polymerase proceeds from matched 3′-primer termini, but does not proceed where a mismatch occurs. Allele specific PCR can be performed in the presence of two different allele-specific primers, one specific for each allele, where each primer is labeled with a different dye, for example one allele specific primer may be labeled with a green dye (e.g., fluorescein) and the other allele specific primer labeled with a red dye (e.g., sulforhodamine). Following amplification, the products are analyzed for green and red fluorescence. The aim is for one homozygous genotype to yield green fluorescence only, the other homozygous genotype to give red fluorescence only, and the heterozygous genotype to give mixed red and green fluorescence.

Methods for performing allele specific PCR are well known in the art, and any such methods may be used. For example suitable methods are taught in Myakishev et al., (2001) Genome Research, 1: 163-169, Alexander et al., (2004) Mol. Biotechnol. 28: 171-174, and Ruano et al., (1989) Nucleic Acids Res. 17: 8392, the contents of which are incorporated by reference. To perform, allele specific PCR the reaction conditions must be carefully adjusted such that the allele specific primer will only bind to one allele and not the alternative allele, for example, in some embodiments the conditions are adjusted so that the primers will only bind where there is a 100% match between the primer sequence and the DNA, and will not bind if there is a single nucleotide mismatch.

The detection of the polymorphism at nucleotide position −1021 of the promoter region of the Dbh gene can be performed using oligonucleotide probes that bind or hybridize to the DNA. These probes may be oligonucleotide fragments. Such fragments should be of sufficient length to provide specific hybridization to the nucleic acid sample. The sequences typically will be about 8 to about 50 nucleotides, but may be longer. Nucleic acid probes having contiguous stretches of about 8, 9, 10, 11, 12, 13, 14, 15, 16, 17, 18, 19, 20, 21, 22, 23, or 24 nucleotides from a region of the Dbh locus) GenBank Accession Nos: AC000404 and AC001227).

The probe sequence must span the particular nucleotide position −1021 of the promoter region of the Dbh gene polymorphism to be detected. For example, probes designed for detection of the −1021 CT Dbh polymorphism must span nucleotide position nucleotide position −1021 of the promoter region of the Dbh gene.

These probes will be useful in a variety of hybridization embodiments, such as Southern blotting, Northern blotting, and hybridization disruption analysis. Also the probes of the disclosure can be used to detect the −1021 CT Dbh polymorphism in amplified sequences, such as amplified PCR products generated using the primers described above. A target nucleic acid may be first amplified, such as by PCR or strand displacement amplification (SDA), and the amplified double stranded DNA product is then denatured and hybridized with a probe.

Double stranded DNA (amplified or not) may be denatured and hybridized with a probe of the present disclosure and then the hybridization complex is subjected to destabilizing or disrupting conditions. By determining the level of disruption energy required wherein the probe has different disruption energy for one allele as compared to another allele, the genotype of a gene at a polymorphic locus can be determined. In one example, there can be lower disruption energy, e.g., melting temperature, for an allele that harbors a cytosine residue at a polymorphic locus, and a higher required energy for an allele with a thymine residue at that polymorphic locus. This can be achieved where the probe has 100% homology with one allele (a perfectly matched probe), but has a single mismatch with the alternative allele e.g., the −1021 CT Dbh polymorphism. Since the perfectly matched probe is bound more tightly to the target DNA than the mis-matched probe, it requires more energy to cause the hybridized probe to dissociate.

The destabilizing conditions may comprise an elevation of temperature: the higher the temperature, the greater the degree of destabilization. In another embodiment, the destabilizing conditions comprise subjecting the hybridization complex to a temperature gradient, whereby, as the temperature is increased, the degree of destabilization increases. In an alternative embodiment, the destabilizing conditions comprise treatment with a destabilizing compound, or gradient comprising increasing amounts of such a compound. Suitable destabilizing compounds include, but are not limited to, salts and urea. Methods of destabilizing or denaturing hybridization complexes are well known in the art, and any such method may be used in accordance with the present disclosure. For example, methods of destabilizing or denaturing hybridization complexes are taught by Sambrook et al., Molecular Cloning; A Laboratory Manual 2d ed. (1989).

For optimal detection of single-base pair mismatches, it is preferable that there is about a 1 degree C. to about a 10 degree C. difference in melting temperature of the probe DNA complex when bound to one allele as opposed to the alternative allele at the polymorphic site. Thus, when the temperature is raised above the melting temperature of a probe:DNA duplex corresponding to one of the alleles, that probe will disassociate.

In other embodiments, two different “allele-specific probes” can be used for analysis of a SNP, a first allele-specific probe for detection of one allele, and a second allele-specific probe for the detection of the alternative allele. For example, in one embodiment the different alleles of the polymorphism can be detected using two different allele-specific probes, one for detecting the −1021 CT Dbh polymorphism, and another for detecting the TT-containing allele (wild-type) at nucleotide position −1021.

Whichever probe sequences and hybridization methods are used, one skilled in the art can readily determine suitable hybridization conditions, such as temperature and chemical conditions. Such hybridization methods are well known in the art. For example, for applications requiring high selectivity, one will typically desire to employ relatively stringent conditions for the hybridization reactions, e.g., one will select relatively low salt and/or high temperature conditions, such as provided by about 0.02 M to about 0.10 M NaCl at temperatures of about 50 degree C. to about 70 degree C. Such high stringency conditions tolerate little, if any, mismatch between the probe and the template or target strand, and are particularly suitable for detecting specific SNPs according to the present disclosure. It is generally appreciated that conditions can be rendered more stringent by the addition of increasing amounts of formamide. Other variations in hybridization reaction conditions are well known in the art (see for example, Sambrook et al., Molecular Cloning; A Laboratory Manual 2d ed. (1989)).

Oligonucleotide sequences used as primers or probes for use in the methods of the present disclosure may be labeled with a detectable moiety. As used herein the term “sensors” refers to such primers or probes labeled with a detectable moiety. Various labeling moieties are known in the art. Said moiety may be, for example, a radiolabel (e.g., ³H, ¹²⁵I, ³⁵S, ¹⁴C, ³²P, etc.), detectable enzyme (e.g., horse radish peroxidase (HRP), alkaline phosphatase etc.), a fluorescent dye (e.g., fluorescein isothiocyanate, Texas red, rhodamine, Cy3, Cy5, Bodipy, Bodipy Far Red, Lucifer Yellow, Bodipy 630/650-X, Bodipy R6G-X and 5-CR 6G, and the like), a colorimetric label such as colloidal gold or colored glass or plastic (e.g., polystyrene, polypropylene, latex, etc.), beads, or any other moiety capable of generating a detectable signal such as a colorimetric, fluorescent, chemiluminescent or electrochemiluminescent (ECL) signal.

Primers or probes may be labeled directly or indirectly with a detectable moiety, or synthesized to incorporate the detectable moiety. In one embodiment, a detectable label is incorporated into a nucleic acid during at least one cycle of a cyclic polymerase-mediated amplification reaction. For example, polymerases can be used to incorporate fluorescent nucleotides during the course of polymerase-mediated amplification reactions. Alternatively, fluorescent nucleotides may be incorporated during synthesis of nucleic acid primers or probes. To label an oligonucleotide with the fluorescent dye, one of conventionally-known labeling methods can be used (e.g., (1996) Nature Biotechnology, 14, 303-308; (1997) Appl. Environ. Microbiol., 63, 1143-1147; (1996) Nuc. Acids Res. 24, 4532-4535). An advantageous probe is one labeled with a fluorescent dye at the 3′ or 5′ end and containing G or C as the base at the labeled end. If the 5′ end is labeled and the 3′ end is not labeled, the OH group on the C atom at the 3′-position of the 3′ end ribose or deoxyribose may be modified with a phosphate group or the like although no limitation is imposed in this respect.

Spectroscopic, photochemical, biochemical, immunochemical, electrical, optical or chemical means can be used to detect such labels. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis. In other embodiments the detection may be via conductivity differences between concordant and discordant sites, by quenching, by fluorescence perturbation analysis, or by electron transport between donor and acceptor molecules.

Detection may be via energy transfer between molecules in the hybridization complexes in PCR or hybridization reactions, such as by fluorescence energy transfer (FET) or fluorescence resonance energy transfer (FRET). In FET and FRET methods, one or more nucleic acid probes are labeled with fluorescent molecules, one of which is able to act as an energy donor and the other of which is an energy acceptor molecule. These are sometimes known as a reporter molecule and a quencher molecule respectively. The donor molecule is excited with a specific wavelength of light for which it will normally exhibit a fluorescence emission wavelength. The acceptor molecule is also excited at this wavelength such that it can accept the emission energy of the donor molecule by a variety of distance-dependent energy transfer mechanisms. Generally the acceptor molecule accepts the emission energy of the donor molecule when they are in close proximity (e.g., on the same, or a neighboring molecule). FET and FRET techniques are well known in the art, and can be readily used to detect the polymorphisms of the present disclosure. See for example U.S. Pat. Nos. 5,668,648, 5,707,804, 5,728,528, 5,853,992, and 5,869,255 (for a description of FRET dyes), Tyagi et al., (1996) Nature Biotech. 14: 303-8, and Tyagi et al., (1998) Nature Biotech. 16: 49-53 (for a description of molecular beacons for FET), and Mergny et al., (1994) Nuc. Acid Res. 22: 920-928, and Wolf et al., (1988) Proc. Natl. Acad. Sci. USA 85: 8790-94 (for general descriptions and methods fir FET and FRET), each of which is hereby incorporated by reference.

One aspect of the present disclosure, therefore, encompasses methods of treating a stimulant addiction of a patient comprising: administering to a patient in need of treatment for stimulant addiction a therapeutically effective dose of a composition comprising a selective dopamine .beta.-hydroxylase inhibitor, wherein the therapeutic dose induces aversion for the stimulant in the patient.

In embodiments of this aspect of the disclosure, the selective dopamine .beta.-hydroxylase inhibitor may be a compound having a formula selected from Formulas I, II, III, IV, (S)-5,7,-difluoro-1,2,3,4-tetrahydronapthalen-2-ylamine and nepicastat (S-5-aminomethyl-1-(5,7-difluoro-1,2,3,4-tetrahydronaphth-2-yl)-1,3-dihydroimidazole-2-thione hydrochloride), or a derivative thereof, or a pharmaceutically acceptable salt thereof.

In one embodiment of this method of the disclosure, the composition may comprise the selective dopamine beta-hydroxylase inhibitor nepicastat (S-5-amino methyl-1-(5,7-difluoro-1,2,3,4-tetrahydronaphth-2-yl)-1,3-dihydroimidazole-2-thione hydrochloride).

In embodiments of the methods of the disclosure, the composition administered to the patient may further comprise a pharmaceutically acceptable carrier or excipient.

This aspect of the disclosure advantageously provides methods of treating a stimulant addiction of a patient, wherein the patient is addicted to cocaine or a derivative thereof, or to an amphetamine or a derivative thereof. In one embodiment of the methods, the stimulant addiction is cocaine addiction.

The present disclosure also encompasses methods of generating abstinence from an addictive compound comprising administering to a patient having an addiction to a stimulant, an amount of a therapeutic composition comprising a selective dopamine beta-hydroxylase inhibitor, wherein the amount administered is effective in generating a response in the recipient patient such that the recipient develops an aversion to the intake of the cocaine or derivative thereof.

In these methods of the disclosure, the patient may have an addiction to cocaine or a derivative thereof, to an amphetamine or a derivative thereof, or to a combination of like addictions.

In one embodiment of the disclosure, the stimulant addiction is cocaine addiction or addiction to a derivative thereof.

In embodiments of this method of the disclosure, the selective dopamine beta-hydroxylase inhibitor can be, but is not limited to, nepicastat.

The present disclosure further encompasses methods of treating a stimulant addiction of a patient, wherein the therapeutically effective dose administered to the patient is selected by: determining the genetic profile of a patient with respect to the gene encoding dopamine beta-hydroxylase, wherein the genetic profile correlates to the level of dopamine beta-hydroxylase activity in the patient; and determining a therapeutically effective dosage of a selective dopamine beta-hydroxylase inhibitor according to the genetic profile of the dopamine beta-hydroxylase encoding gene.

In embodiments of the methods of this aspect of the present disclosure, when the patient is homozygous negative for dopamine beta-hydroxylase, the therapeutically effective dose administered to the patient may be advantageously less than if the patient has at least one dopamine beta-hydroxylase positive allele. For example, a C-T transition at nucleotide position −1021 within the promoter region of the Dbh locus would indicate that a lower effective dose of the DBH inhibitor was likely necessary whereas, in the absence of the polymorphism variant, a higher dose should be administered to the addicted patient.

Another aspect of the disclosure, therefore, encompasses methods of selecting a therapeutic dose of a composition for treatment of a patient having a stimulant addiction comprising: determining the genetic profile of a patient with respect to a gene encoding dopamine beta-hydroxylase, wherein the genetic profile correlates to the level of dopamine beta-hydroxylase activity in the patient; and determining a therapeutically effective dosage of a selective dopamine beta-hydroxylase inhibitor according to the genetic profile of the dopamine beta-hydroxylase encoding gene.

In embodiments of this aspect of the disclosure, the selective dopamine beta-hydroxylase inhibitor may comprise nepicastat or a derivative thereof.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims. The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in degree C., and pressure is at or near atmospheric. Standard temperature and pressure are defined as 20 degree C. and 1 atmosphere.

### EXAMPLES

### Example 1

Cocaine-induced locomotion in Dbh −/− mice: If selective DBH inhibition is therapeutic for cocaine dependence, then DBH knockout (Dbh −/−) mice that completely lack DBH protein would be expected to have altered responses to psychostimulants. The locomotor response of Dbh +/− and Dbh −/− mice to amphetamine and cocaine was measured, and it was found that Dbh −/− mice were hypersensitive to both psychostimulant-induced locomotion and stereotypy (Weinshenker et al., 2002; Schank et al., 2005).

Locomotor activity measurement: Experiments were conducted in an isolated behavior room between 1000 and 1600 hrs. Ambulations (consecutive beam breaks) were measured in transparent plexiglass cages (40 times 20 times 20 cm³) placed into a rack with seven infrared photobeams spaced 5 cm apart, each end beam 5 cm from the cage wall (San Diego Instruments Inc., LaJolla, Calif.). Mice were placed in the activity chambers for 4 hr, injected with cocaine (5, 10, or 20 mg/kg i.p.; Sigma-Aldrich, St Louis, Mo.), and ambulations were recorded for an additional 2 hr. Data were analyzed by ANOVA followed by Bonferroni post-hoc tests. For the antagonist studies, saline, the 5-HT_{1A} antagonist WAY100635 (0.03 mg/kg), the 5-HT₂ antagonist ketanserin (0.3 mg/kg), the D₁ antagonist SCH23390 (0.03 mg/kg), or the D₂ antagonist eticlopride were injected i.p. 30 min prior to cocaine (20 mg/kg). Antagonist doses were chosen based on the literature and our pilot experiments; higher doses were tried, but typically resulted in sedation and ataxia, indicating nonspecific effects. All drugs were purchased from Sigma-Aldrich (St Louis, Mo.).

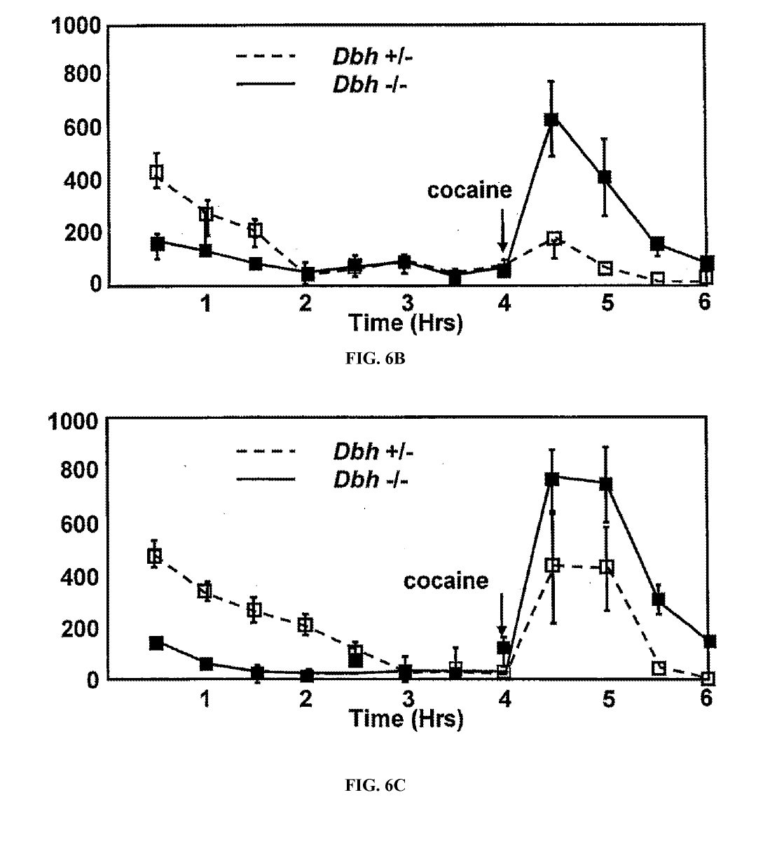

The effects for cocaine are shown in FIGS. 6A-6C. Mice were placed in activity chambers and injected with cocaine 4 hours later with 5 mg cocaine/kg (FIG. 6A), 10 cocaine mg/kg (FIG. 6B), or 20 mg cocaine/kg (FIG. 6C). Ambulations were recorded for 2 additional hours. (Shown is mean SEM. * P<0.05, ** P<0.01, *** P<0.001 compared to Dbh +/− mice).

Cocaine produced a dose-dependent increase in locomotor activity in both Dbh +/− and −/− mice. However, as with amphetamine, cocaine-induced locomotion was greater in Dbh −/− mice at all doses tested. Locomotor activity in response to a novel environment prior to drug administration is reduced in Dbh −/− mice, as described previously (Weinshenker et al., (2002) Proc. Natl. Acad. Sci. USA 99: 13873-13877) (see first 60 min after placement in activity chambers in FIGS. 6A-6C).

### Example 2

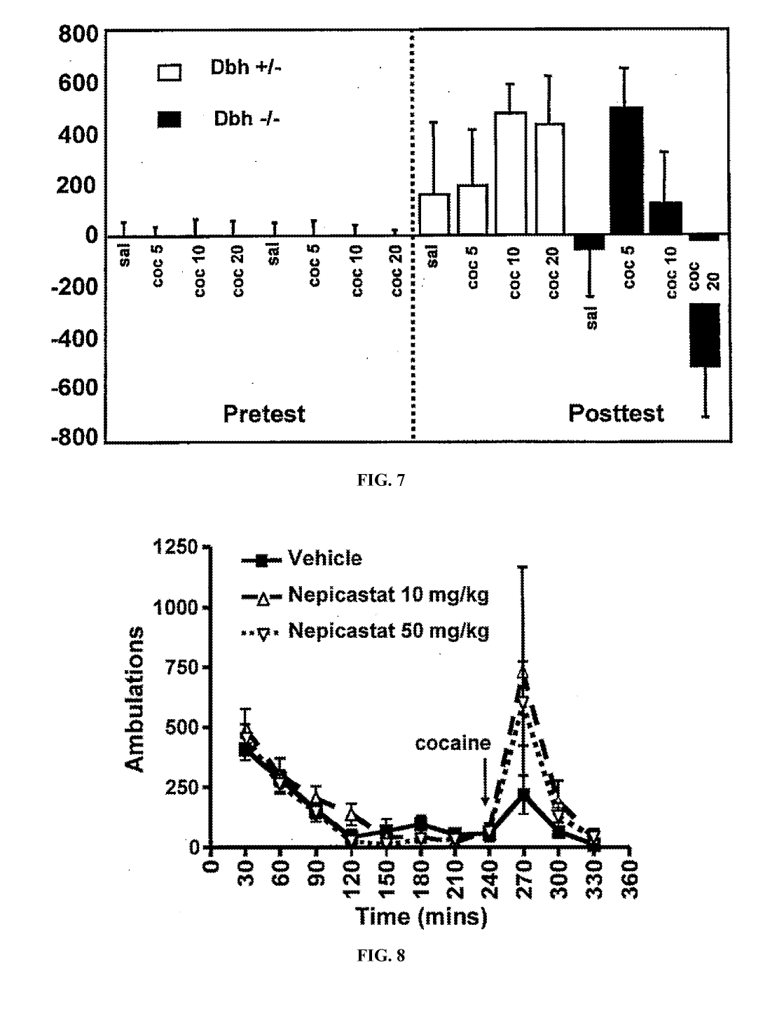

Altered cocaine reward and aversion in Dbh −/− mice: To determine whether lack of DBH alters psychostimulant reward and/or aversion, cocaine-conditioned place preference (CPP) was assessed in Dbh +/− and Dbh mice. The side preference of both genotypes before pairing with cocaine and after pairing with saline was essentially random. While Dbh +/− mice expressed a significant place preference to cocaine at the moderate and high dose (10 and 20 mg/kg) but not the low dose (5 mg/kg), Dbh −/− mice expressed a significant preference only at the low dose and avoided the cocaine-paired chamber at the high dose. FIG. 7 illustrates preference in seconds for the “cocaine-paired” (saline, cocaine 5, 10, or 20 mg/kg) side before (Pretest) and after (Posttest) 3 days of pairing. (N=7-10 per group. Values are expressed as mean SEM. * P<0.05 compared to Pretest for that group.)

The aversion of Dbh −/− mice to a dose of cocaine (20 mg/kg) that produced a place preference in control mice suggests a hypersensitivity to the aversive effects of cocaine. The increased cocaine aversion observed at higher doses of cocaine in Dbh −/− mice may overwhelm the rewarding effects of cocaine, resulting in a failure of Dbh −/− mice to express a conditioned place preference to the two higher doses of cocaine tested.

### Example 3

Using a novel technique to deliver disulfuram via osmotic minipump, mice were exposed to a continuous dose of 50 mg/kg/day for 2 to 3 weeks. It was found that locomotion induced by a dose of 10 mg/kg cocaine was decreased in cocaine-naive animals pretreated with disulfuram minipumps.

Because disulfuram inhibits many enzymes besides DBH, the effect of the highly specific DBH inhibitor, nepicastat, on drug-induced behavior was also tested. Mice were implanted with minipumps for delivering either vehicle (50% DMSO, 0.9% NaCl or nepicastat (10 or 50 mg/kg/d). 3 weeks later, the mice were put in activity chambers, allowed to acclimate for 4 hrs, then injected with cocaine (10 mg/kg, i.p.) and ambulations measured for 2 additional hours, as shown in FIG. 8 (N=6 per group).

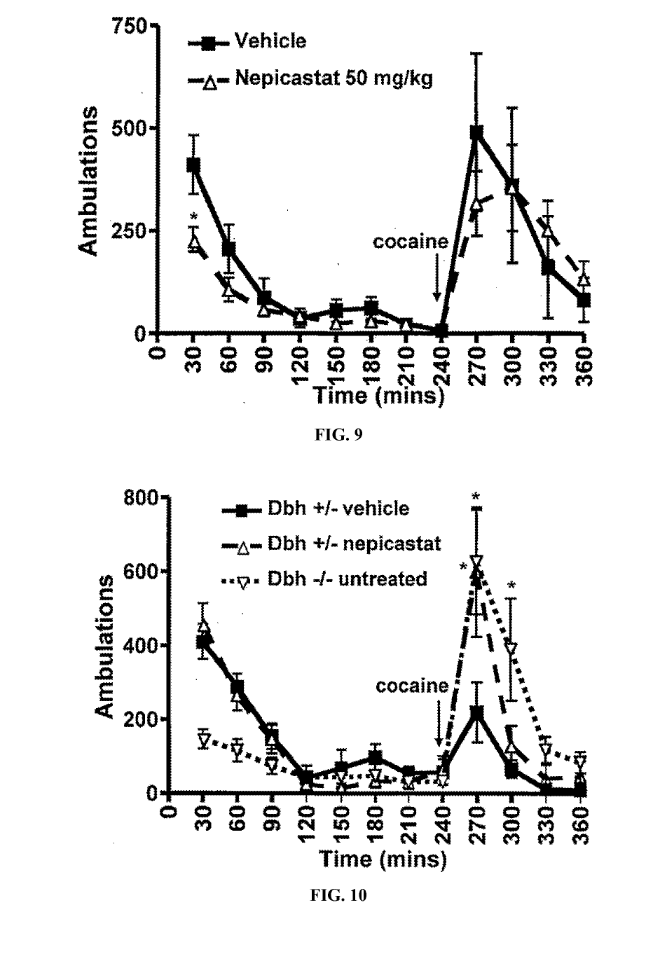

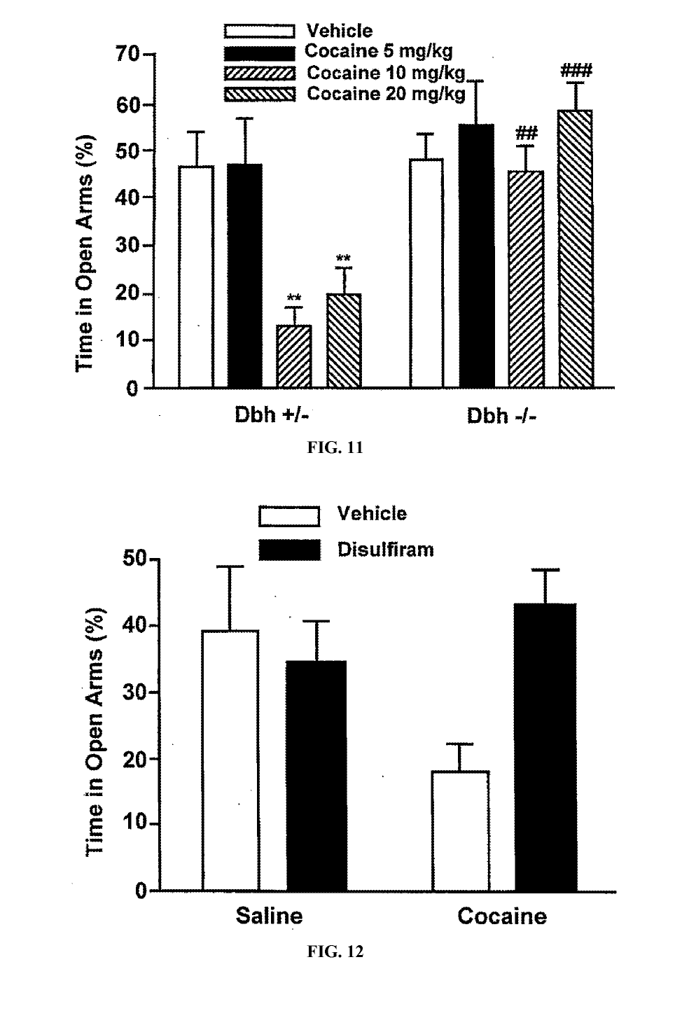

To test whether a single, acute, dose of nepicastat had a different effect than prolonged nepicastat exposure, mice were given a single injection (i.p.) with either vehicle (10% DMSO, 0.9% NaCl) or nepicastat (50 mg/kg). Two hours later, the mice were put in activity chambers, allowed to acclimate for 4 hrs, injected with cocaine (20 mg/kg, i.p.) and ambulations measured for 2 additional hours. (FIG. 9) (N=8 per group).

Results demonstrated that prolonged (e.g., 3 weeks) treatment with 10 or 50 mg/kg/day of nepicastat significantly increased locomotion induced by 10 mg/kg cocaine (FIG. 8). In contrast, a single, acute dose of nepicastat did not significantly affect cocaine-induced locomotion (FIG. 9). This effect of chronic DBH inhibition by nepicastat recapitulates the cocaine hypersensitivity previously observed in DBH knockout mice (FIG. 10). Taken together, these results suggest that chronic pharmacological DBH inhibition can alter catecholamine levels and certain drug induced behaviors in rodents. However, there appears to be some distinction between the effects of specific versus non-specific inhibition of this enzyme.

### Example 4

Male and female Dbh +/− and −/− mice (aged 2 to 5 months) were individually housed on a reversed light cycle (lights on at 19:00, lights off at 7:00), and were allowed a minimum of two weeks to habituate to the new lighting conditions after moving from normal light cycle (lights on at 7:00, lights off at 19:00). Food and water were available ad libitum throughout the course of the study. Data from male and female mice were combined, since there were no detectable gender differences. Dbh mice were generated as described (15) and maintained on a mixed C57B16/J and 129SvEv background. Dbh +/− mice were used as controls, because they have normal brain catecholamine levels and are behaviorally identical to wild-type (Dbh +/+) mice (14-16). Three-month old male and female C57BL6/J mice (Jackson Labs, Bar Harbor, Minn.) were also used to generalize the findings from these experiments to a different strain of wild-type mouse. Housing, handling, and testing conditions for these animals were identical to those used in experiments with Dbh +/− mice.

### Example 5

Behavioral testing The EPM apparatus consisted of two open arms and two enclosed arms arranged in a plus orientation. The arms were elevated 30 inches above the floor, with each arm projecting 12 inches from the center. Because rodents naturally prefer dark, enclosed compartments, a greater willingness to explore the open, well-lit arms is believed to represent a decrease in the animal's anxiety. This interpretation has been validated by the efficacy of known anxiolytic and anxiogenic treatments in this paradigm (Paine et al., (2002) Behavioural Pharmacol. 13: 511-5237; Gorman & Dunn (1993) Pharmacol. Biochem. & Behavior 45: 1-7; Pellow et al., (1985) J. Neurosci. Methods 14: 149-167; Johnston & File (1988) Pharmacol. Biochem. & Behavior 32: 151-156).

In all experiments, cocaine was injected 20 minutes prior to behavioral testing as described by Yang and colleagues (Yang et al., (1992) Pharmacol. Biochem. & Behavior 41: 643-650). To begin each test, mice were placed in the EPM facing one of the open arms and allowed to freely explore the apparatus for five minutes, during which time their behavior was videotaped. Videotapes were later scored by an observer who was blind to genotype and treatment group. The measure used for analysis was the percentage of time spent exploring the open arms, which was calculated by dividing the time spent in the open arms by the combined time spent in open and closed arms. Because some drug treatments and genetic manipulations alter overall locomotor activity, it was important to use this percentage measurement as the dependent variable for analysis (Pellow et al., (1985) J. Neurosci. Methods 14: 149-167).

Entry into an arm of the plus maze was defined as the animal placing all four paws in that particular compartment of the apparatus. All tests were run during the dark cycle, between 14:00 and 18:00. Mice were excluded from data analysis for any of the following reasons: if they jumped or fell off the maze after test had begun, if they had a seizure while on the testing apparatus, or if their open arm time was detected as an outlier using Grubb's test. Of 253 total mice tested, 10 were excluded from data analysis. Data were analyzed using independent samples t-tests, one-way ANOVA followed by Dunnett's post-hoc tests, or two-way ANOVA followed by Bonferroni post-hoc tests using Prism 4.0 for Macintosh.

### Example 6

Cocaine dose-response: Dbh +/− and −/− mice (n=8 per group) were injected with 0.9% saline (i.p., 10 ml/kg) or cocaine (5, 10, or 20 mg/kg, i.p. at 10 ml/kg, dissolved in 0.9% saline) 20 minutes prior to behavioral testing. Behavioral testing then proceeded for five minutes, as described in Example 5 above.

Baseline performance on the EPM was similar for Dbh +/− and Dbh −/− mice as shown in FIG. 11). Cocaine treatment dose-dependently decreased percent open arm time in Dbh +/− mice. In contrast, the anxiety behavior of Dbh −/− mice was unaffected by cocaine treatment at any dose (FIG. 11). Two-way ANOVA revealed main effects of dose (F[3,56]=4.391, p=0.0076) and genotype (F[1,56]=19.78, p<0.0001), as well as a dose-genotype interaction (F[3,56]=4.046, p=0.0113). Bonferroni post-hoc analysis indicated a significant decrease in percent open arm time only in Dbh +/− mice treated with 10 mg/kg cocaine (p<0.01) and 20 mg/kg cocaine (p<0.01), when compared to saline treated Dbh +/− animals. Also, Dbh +/− animals showed a lower level of open arm exploration when compared to Dbh mice for doses of 10 mg/kg (p<0.01) and 20 mg/kg cocaine (p<0.001).

### Example 7

DBH inhibition in Dbh +/− mice: DBH enzyme activity was inhibited pharmacologically via acute administration of disulfuram. Disulfuram is a copper-chelating agent that inhibits DBH activity and alters catecholamine tissue content (Bourdelat-Parks et al., (2005) Psychopharmacol. 183(1): 72-80; Maj et al., (1968) J. Pharmacy Pharmacol. 20: 247-248; Musacchio et al., (1966) J. Pharmacol. Experimental Therapeutics 152(1): 56-61).

Mice (n=8 per group) were given three injections, each spaced two hours apart, with either vehicle (0.9% saline) or disulfuram (200 mg/kg, i.p. at 10 ml/kg, sonicated and suspended in 0.9% saline). This dosing regimen is known to decrease NE by about 70% in the mouse brain (Bourdelat-Parks et al., (2005) Psychopharmacol. 183(1): 72-80). Mice received cocaine (10 mg/kg, i.p. at 10 ml/kg, dissolved in 0.9% saline) or saline two hours after the final pretreatment injection, and behavioral testing took place 20 minutes later, as described above. To habituate the mice to the multiple daily injection regimen and large total injection volumes, they were injected three times per day (spaced two hours apart) with 0.9% saline (10 ml/kg) for three days prior to test day.

To determine whether NE depletion confers resistance to cocaine-induced anxiety in normal animals, Dbh +/− mice were pretreated with the DBH inhibitor disulfuram or vehicle prior to cocaine administration and EPM testing. Disulfuram abolished the ability of cocaine to reduce open arm exploration time in Dbh +/− mice, but had no effect in animals treated with saline prior to testing, as shown in FIG. 13.

Two way ANOVA revealed a pretreatment by drug treatment interaction (F[1,28]=5.227, p=0.03). Bonferroni post-hoc tests indicated that the Disulfuram-Cocaine group showed a significantly increased level of open arm exploration relative to the Vehicle-Cocaine group (p<0.05). Temporary inhibition of NE production decreases cocaine-induced anxiety, phenocopying the behavior observed in Dbh mice.

### Example 8

Administration of adrenergic antagonists in Dbh +/− mice: Dbh +/− mice (n=10-17 per group) were pretreated with 0.9% saline (4 ml/kg, i.p.), vehicle (0.9% saline with 1.5% DMSO, 1.5% Cremaphor EL, 10 ml/kg, i.p), the beta-adrenergic receptor (beta-AR) antagonist propranolol (5 mg/kg, i.p. at 4 ml/kg, dissolved in 0.9% saline), the alpha1-AR antagonist prazosin (0.5 mg/kg, i.p. at 10 ml/kg, dissolved in vehicle), or the alpha 2-AR antagonist yohimbine (2.5 mg/kg, i.p. at 10 ml/kg, dissolved in distilled water) 10 minutes prior to cocaine injection. Behavioral testing was then performed 20 minutes after cocaine injection (10 mg/kg, i.p. at 10 ml/kg, dissolved in saline). Open arm times for the saline and vehicle groups were compared, and no differences were found; therefore these two groups were combined to form a single control group.

As shown in Example 6, NE is likely required for the anxiogenic effect of cocaine in the EPM. To determine which subtype of adrenergic receptor is critical for cocaine-induced anxiety, we pretreated Dbh +/− mice with the alpha 1-AR antagonist prazosin, the 2-AR antagonist yohimbine, or the beta-AR antagonist propranolol prior to administration of cocaine and EPM testing. Cocaine induced anxiety was preserved following prazosin or yohimbine treatment, but abolished by propranolol, as shown in FIG. 4.

One way ANOVA revealed a significant effect of antagonist treatment on percent open arm time (F[3,63]=3.485, p=0.0211), and Dunnett's post-hoc tests indicated that the propranolol group differed significantly from the control group (p<0.01), whereas the prazosin and yohimbine groups did not, indicating that NE signaling through alpha-adrenergic receptors is required for the cocaine-induced anxiety behavior of mice as measured by the EPM.

To examine the possibility that the pretreatments alone can alter plus maze behavior, Dbh +/− mice were treated with either propranolol, prazosin, or yohimbine, at the same doses considered above, and tested plus maze behavior 20 minutes later. This data was then compared to behavior observed in saline-treated Dbh +/− mice, and one-way ANOVA revealed no significant effect of antagonist treatment on percent open time (F[3,31]=1.892, p=0.1539, n=7-10 per group).

### Example 9

Beta-adrenergic inhibition in C57BL6/J wild type mice: C57BL6/J wild type mice (n=7 per group) were pretreated with 0.9% saline (10 ml/kg, i.p.) or propranolol (5 mg/kg, i.p. at 10 ml/kg, dissolved in 0.9% saline) 10 minutes prior to injection of saline or cocaine (10 or 20 mg/kg, i.p.). Behavioral testing was performed 20 minutes following cocaine injection, as described above. In preliminary experiments with this mouse strain, the 10 mg/kg dose of cocaine was not sufficient to induce significant cocaine-induced anxiety. Therefore, data for the 20 mg/kg dose is reported.

To assess whether the results could be generalized to other wild-type mouse strains, the effect of propranolol pretreatment was tested on cocaine-induced anxiety behavior in pure C57BL6/J mice. Similar to our previous results, cocaine reduced percent open arm time, and this effect was blocked by propranolol. Propranolol had no effect on baseline performance. Two way ANOVA revealed a main effect of drug treatment (F[1,24]=5.694, p=0.0253). Bonferroni post-hoc tests indicated that only percent open arm time of the Vehicle-Cocaine group was significantly lower than the Vehicle-Saline group (p<0.05).

1. A method of treating cocaine addiction comprising administering a pharmaceutical composition comprising S-5-aminomethyl-1-(5,7-difluoro-1,2,3,4-tetrahydronaphthalen-2-yl)-1,3-dihydroimidazole-2-thione or salt thereof to a patient diagnosed with cocaine addiction.

2. The method of claim 1, wherein the salt is a hydrochloride salt.

3. The method of claim 1, wherein the patient is administered about 7 mg to about 350 mg of S-5-aminomethyl-1-(5,7-difluoro-1,2,3,4-tetrahydronaphthalen-2-yl)-1,3-dihydroimidazole-2-thione hydrochloride daily.

4. The method of claim 1, wherein the patient is a human.

5. The method of claim 4, wherein the patient is tested for a C or T at nucleotide position 1021 in the Dbh gene.
